(19) United States
(12) Patent Application Publication
(10) Pub. No.: US 2019/0118174 A1
(43) Pub. Date: Apr. 25, 2019

(54) MICROFLUIDICS FOR ANALYTE DETECTION BASED ON THE LIGHT TO HEAT CONVERSION PROPERTIES OF METAL NANOPARTICLES

(71) Applicant: NANOIMMUNOTECH, S.L., Vigo (ES)

(72) Inventors: Mariantonietta PARRACINO, Vigo (ES); José María ABAD PASTOR, Vigo (ES); Sara PUERTAS LORENTE, Vigo (ES); Valeria GRAZU BONAVIA, Vigo (ES); Jesús MARTÍNEZ DE LA FUENTE, Zaragoza (ES); Ana Luisa DO VALE FONSECA CLARO, Zaragoza (ES)

(21) Appl. No.: 16/089,256

(22) PCT Filed: Mar. 28, 2017
(86) PCT No.: PCT/EP2017/057346

§ 371 Date: Sep. 27, 2018

(30) Foreign Application Priority Data
Mar. 28, 2016 (EP) 16382137.4

(51) Int. Cl. B01L 3/00; G01N 21/17; G01N 33/543; G01N 33/58

(52) U.S. Cl. CPC B01L 3/502707; G01N 21/171; B82Y 15/00; G01N 33/587; G01N 33/54373

(57) ABSTRACT

The present invention refers to the in vitro use of a microfluidic kit or device comprising a support or substrate, wherein said support or substrate comprises at least one channel in the substrate, the channel comprising an inlet, an outlet, and a flow-path connecting the inlet and outlet, wherein the inlet and outlet together define a midplane; and a portion of the flowpath travels transversely across the midplane, wherein the portion of the flowpath that travels transversely across the midplane includes a recognition site or sensing area for detecting a target analyte; for detecting an analyte as a result of the heat generated by metal nanoparticles when they are irradiated with an external light source.

### FIELD OF THE INVENTION

The present invention refers to the field of microfluidics, in particular it shows that microfluidic chips are especially suitable for use in a number of immunoassays (such as ELISA immunoassays) for detecting an analyte as a result of the heat generated by metal nanoparticles when they are irradiated with an external light source.

### BACKGROUND OF THE INVENTION

There is an increase of the market of biosensing in the agro-food sector, where the ultrasensitive and low cost detection of food contaminants is required.

In particular, the companies in the poultry industry perform routine control tests for the presence/absence of pathogens such as Salmonella, E. coli or Campylobacter wherein the detection protocol is broadly regulated in the meat itself as well as in boot swabs, work boots, work tables, poultry fattening, laying hen farms, etc.

For the specific detection of Salmonella, different methods have been developed, based on an immunoassay such as ELISA, or on other suitable assays such as PCR and stock culture, to reduce the time required for the detection of this pathogen, because standard culture methods, such as the International Organization for Standardization Method 6579 (ISO) and the United States Food and Drug Administration's Bacteriological Analytical Manual Chapter 5: Salmonella (FDA), although they have a very low detection limit of 9 CFUs/mL (colony forming units per mL) for both poultry meat and poultry meat products, require up to 5 days (including biochemical and serological confirmations; ISO, 2002; FDA, 2007) to finalize the methods, and are thus not efficient in the routine monitoring of large numbers of samples. In this context, rapid, accurate, and economical methods, are crucial both for the industry and for laboratories reporting results to governmental authorities for taking legal actions. One of these methods is the Vitek immunodiagnostic assay (VIDAS; Biomérieux, Marcy L'Etoile, France), an automated enzyme-linked fluorescent assay-based system that allows for the accurate and rapid screening of large numbers of samples for the presence of Salmonella by the Vitek immunodiagnostic assay system Salmonella (VIDAS SLM) method. The detection limit of VIDAS ESLM for both poultry meat and poultry meat products, was determined to be 90 cfu/mL, in 48 hours.

However, to date all known tests, including Vitek immunodiagnostic assay, for screening samples for the presence of Salmonella require qualified staff and specific laboratory equipment, significantly delaying the provision of the results. If we take into account the fact that Salmonella-positive result in any of the known tests may imply the slaughter of all chickens in a housing unit, unless it can be treated early, it is a major issue for the food industry to identify the presence of pathogens as quickly and efficiently as possible in order to take the appropriate measures.

The present invention provides a rapid, highly sensitive and specific method for the identification of a wide variety of analytes, including pathogens such as Salmonella, E. coli or Campylobacter, in an efficient manner.

### BRIEF DESCRIPTION OF THE INVENTION

Disclosed herein, in various exemplary embodiments, we show that microfluidic chips are especially suitable for use in a number of immunoassays for detecting an analyte as a result of the heat generated by metal nanoparticles when they are irradiated with an external light source.

These devices are useful for detecting the presence of one or more target analytes in one or more sample fluids. Methods and processes of making and using such devices are also disclosed in the examples.

Therefore, in particular the present invention refers to the in vitro use of a microfluidic kit or device comprising a support or substrate, wherein said support or substrate comprises at least one channel in the substrate, the channel comprising an inlet, an outlet, and a flow-path connecting the inlet and outlet, wherein the inlet and outlet together define a midplane, and a portion of the flowpath travels transversely across the midplane, wherein the portion of the flowpath that travels transversely across the midplane includes a recognition site or sensing area for detecting a target analyte;

for detecting an analyte as a result of the heat generated by metal nanoparticles when they are irradiated with an external light source.

It is noted that midplane is a plane passing through the channel in such a way as to divide it into symmetrical halves and sensing area is defined as the portion of the metal-chetale activated surface functionalized with the antibody, identified inside the flowpath that travels transversely across the midplane between the inlet and outlet.

### BRIEF DESCRIPTION OF THE INVENTION

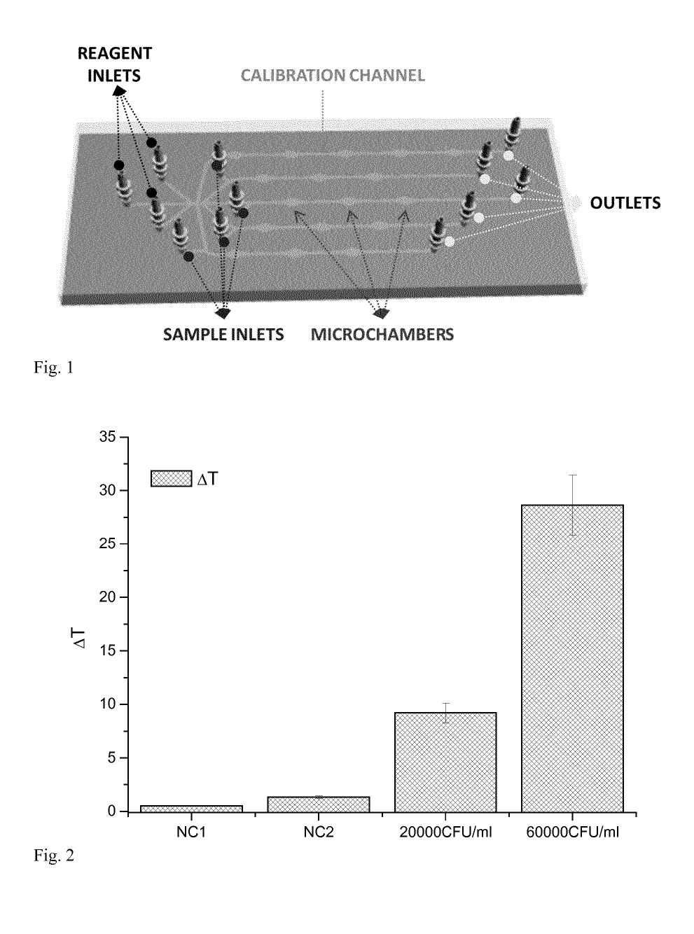

FIG. 1. Microfluidic prototype. NC=negative control; PC: positive control.

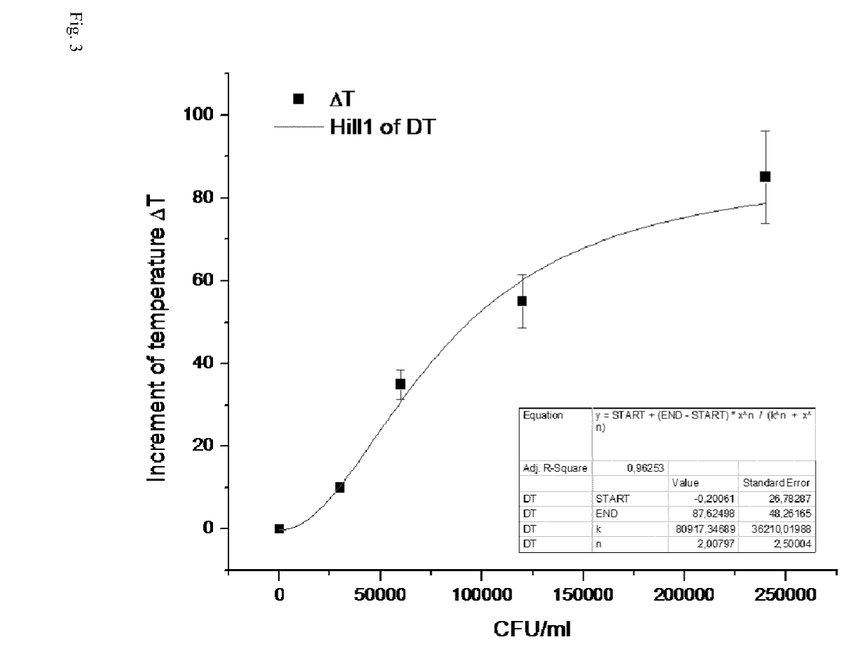

FIG. 2. Measurement of the increment of temperature for two different concentrations of salmonella diluted in buffer and directly adsorbed onto a microfluidic chamber, upon irradiation with 30 mW NIR beam.

FIG. 3. Measurement of the increment of temperature AT of salmonella T. at different concentrations, directly adsorbed onto the surface of microfluidic chip.

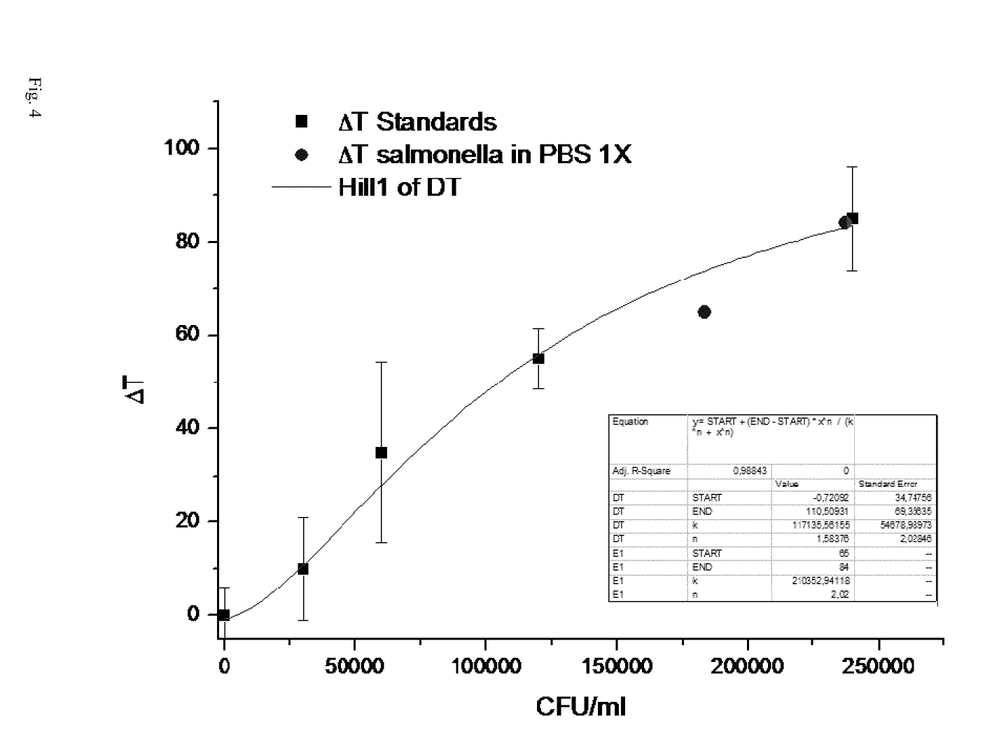

FIG. 4. Measurement of the increment of temperature AT due to the presence of salmonella T. at different concentrations, detected with sandwich immunoassay onto the surface of microfluidic chip.

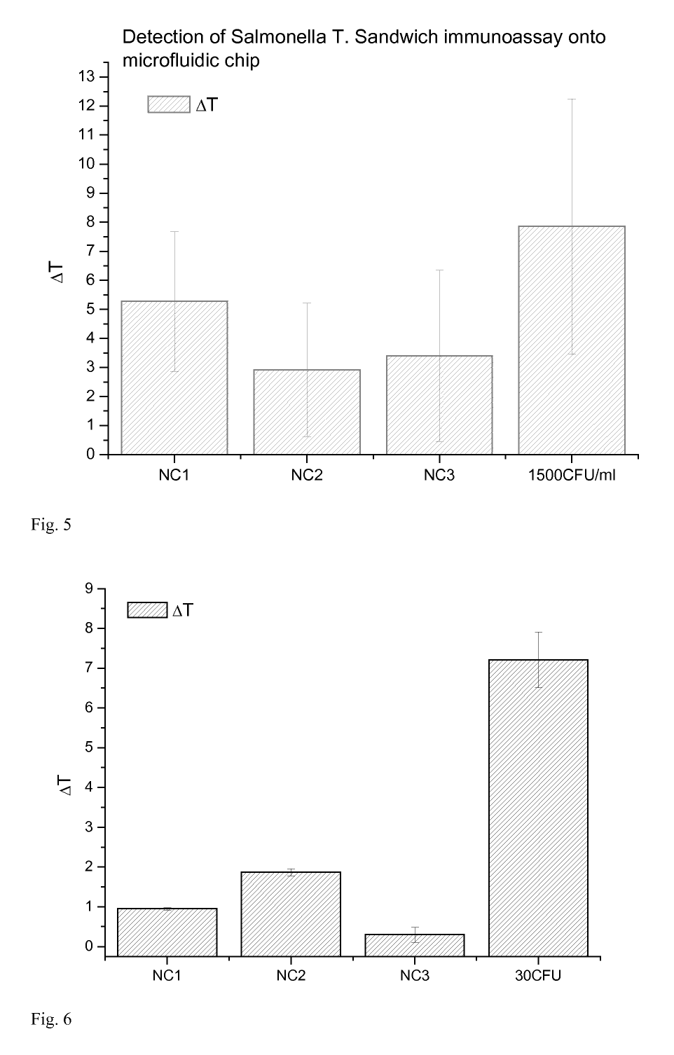

FIG. 5. Measurement of the increment of temperature due to the immunodetection of one concentration of salmonella, diluted in buffer phosphate, in a sandwich format, onto the microfluidic chip, upon irradiation with 30 mW NIR laser beam.

FIG. 6. low limit of detection of salmonella on Ab adsorbed onto the surface of microfluidic chip.

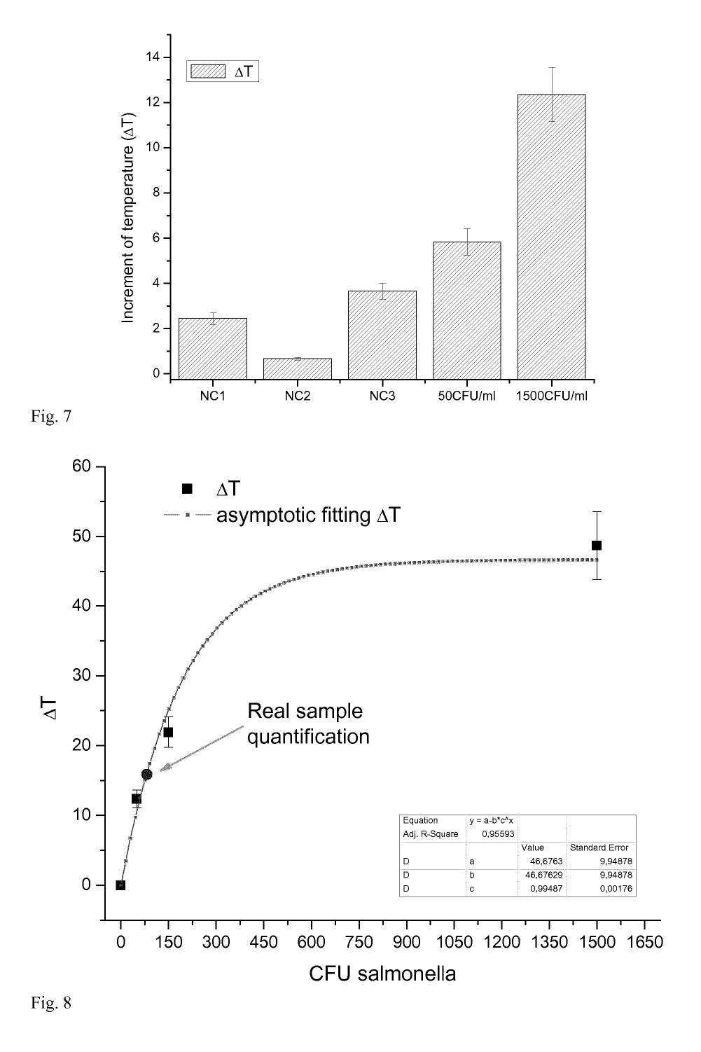

FIG. 7. Measurement of the increment of temperature DT, due to the presence of salmonella in real sample doped with two dilution of it upon irradiation with 30 mW NIR laser beam.

FIG. 8. Quantification of the real sample from the calibration curve.

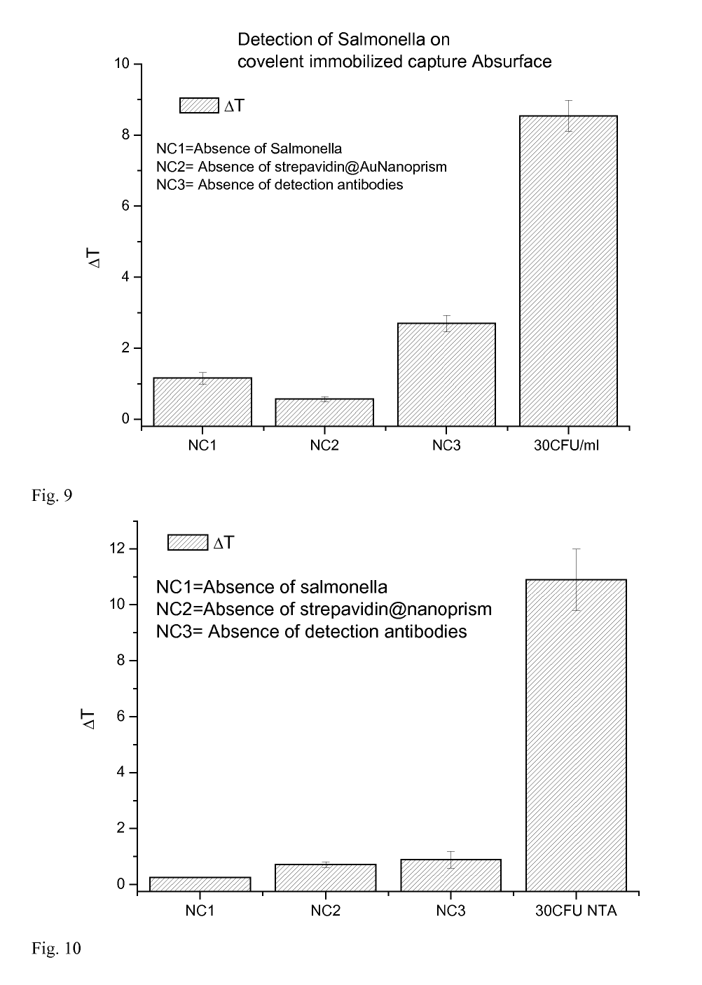

FIG. 9. Measurement of the increment of temperature AT, due to the presence of salmonella in PBS doped with 30 CFU/ml, using a covalent immobilized capture antibodies of it upon irradiation with 30 mW NIR laser beam.

FIG. 10. Measurement of the increment of temperature AT, due to the presence of salmonella in PBS doped with 30 CFU/ml, using a oriented immobilized capture antibodies of it upon irradiation with 30 mW NIR laser beam.

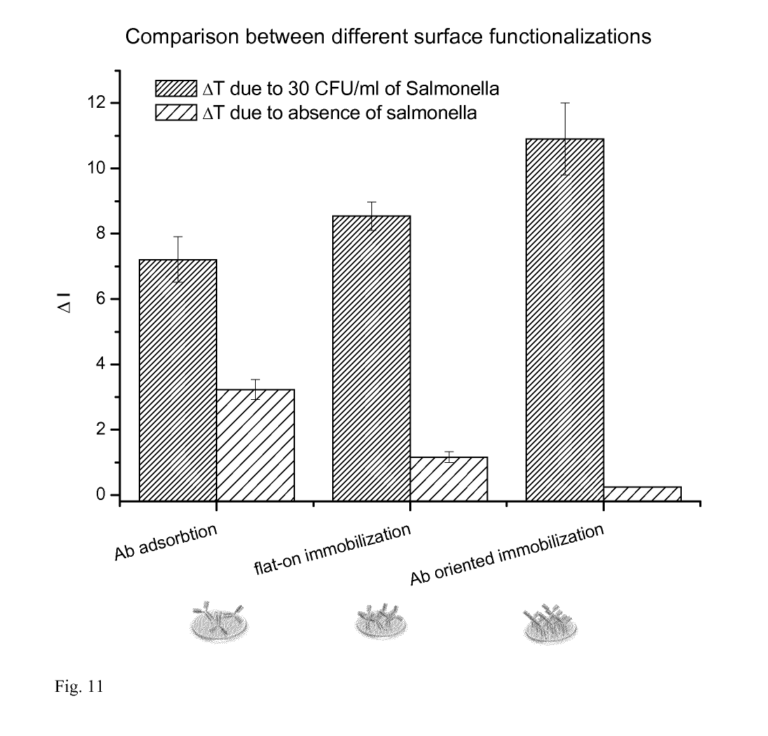

FIG. 11. Comparison between different surface functionalizations of the microfluidic chip surface.

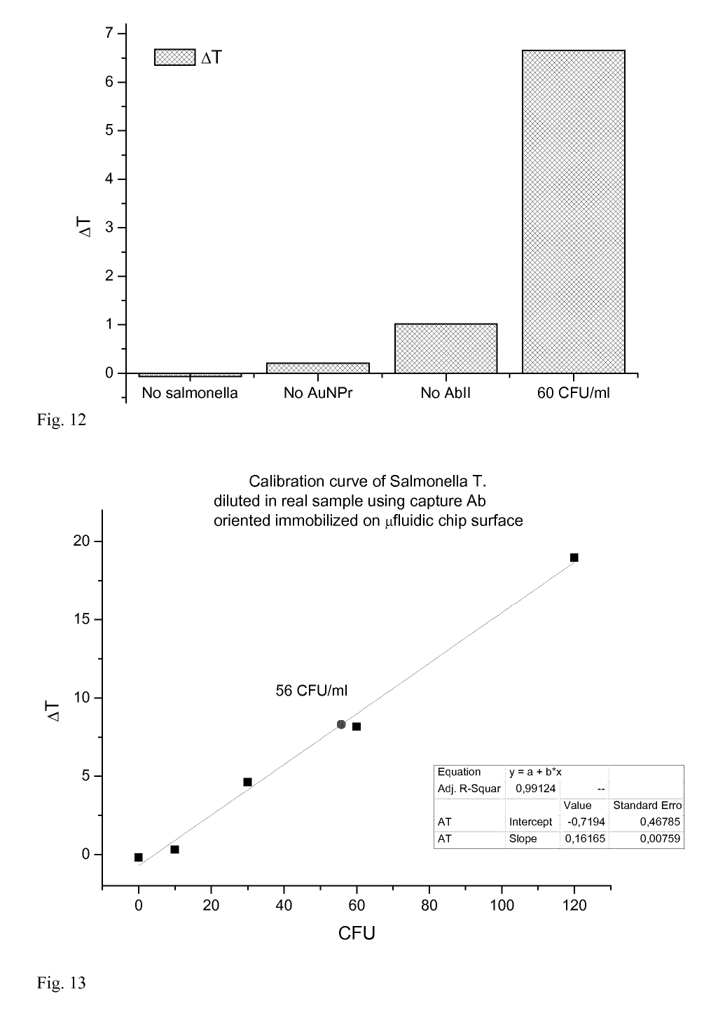

FIG. 12. Detection of 60 CFU/ml of salmonella in a real sample of 25 g of chicken meat in 225 ml of peptone.

FIG. 13. Quantification of Salmonella in real sample (in a real sample of 25 g of chicken meat in 225 ml of peptone) onto oriented capture antibodies functionalized microfluidic chip.

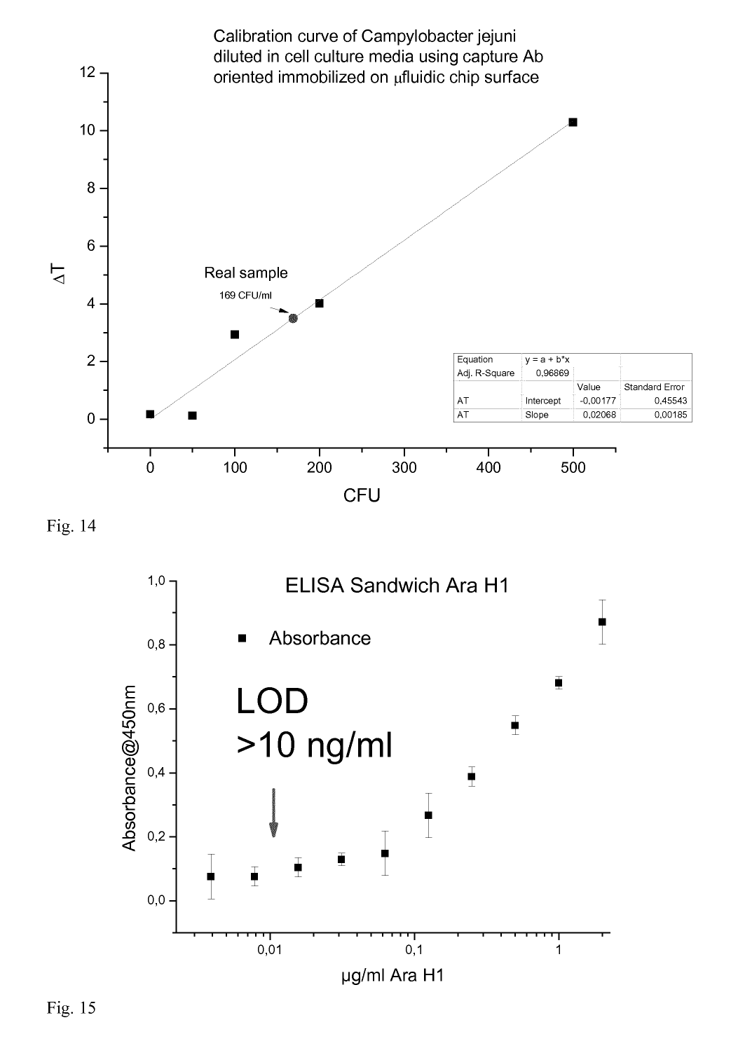

FIG. 14. Quantification of Campylobacter jejuni in Bolton culture media onto oriented capture antibodies functionalized microfluidic chip.

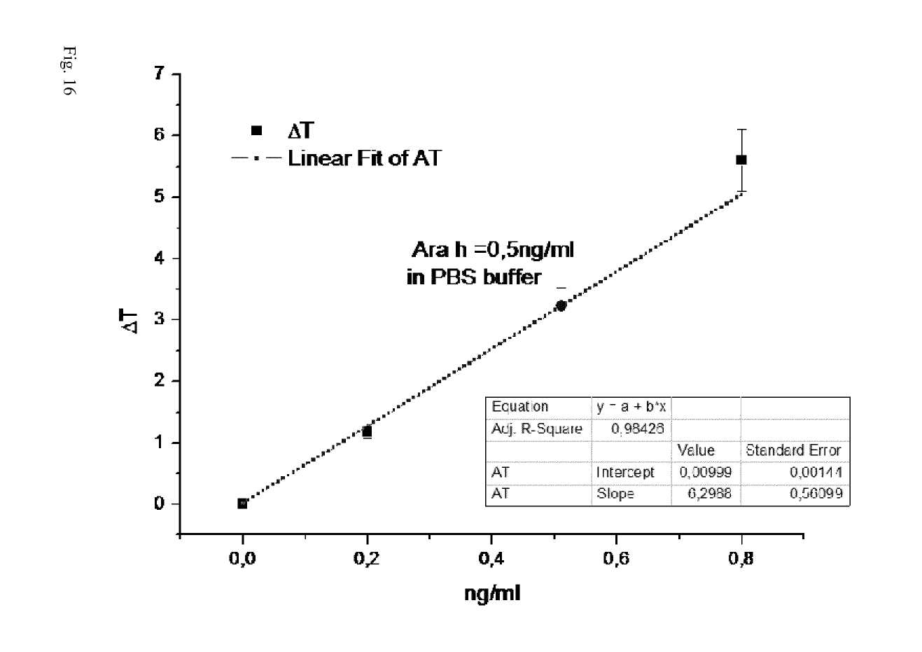

FIG. 15. Determination of LOD of Ara h1 using a commercial available ELISA kit (Ara h 1 ELISA kit (EL-AH1) Ara h 1 ELISA kit (2C12/2F7) from Indoor Biotechnology, www.inbio.com).

FIG. 16. Quantification of Ara h 1 in real sample onto oriented capture antibodies functionalized microfluidic chip.

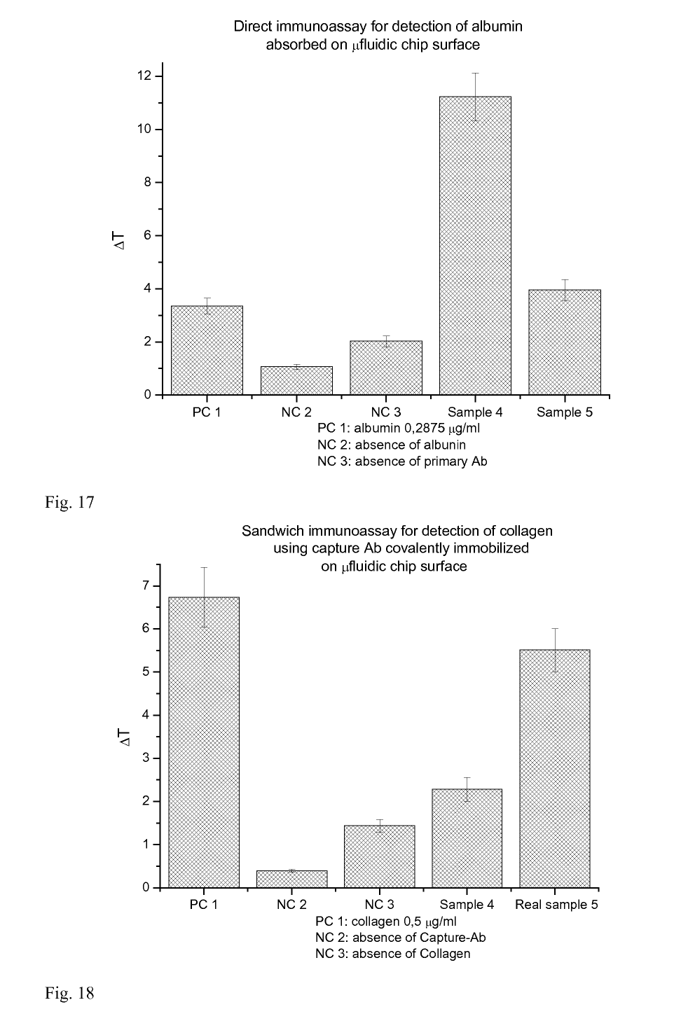

FIG. 17. Direct immunoassay for detection of albumin absorbed on microfluidic chip chamber surface.

FIG. 18. Sandwich immunoassay for detection of collagen using capture antibodies covalently immobilized on microfluidic chip surface

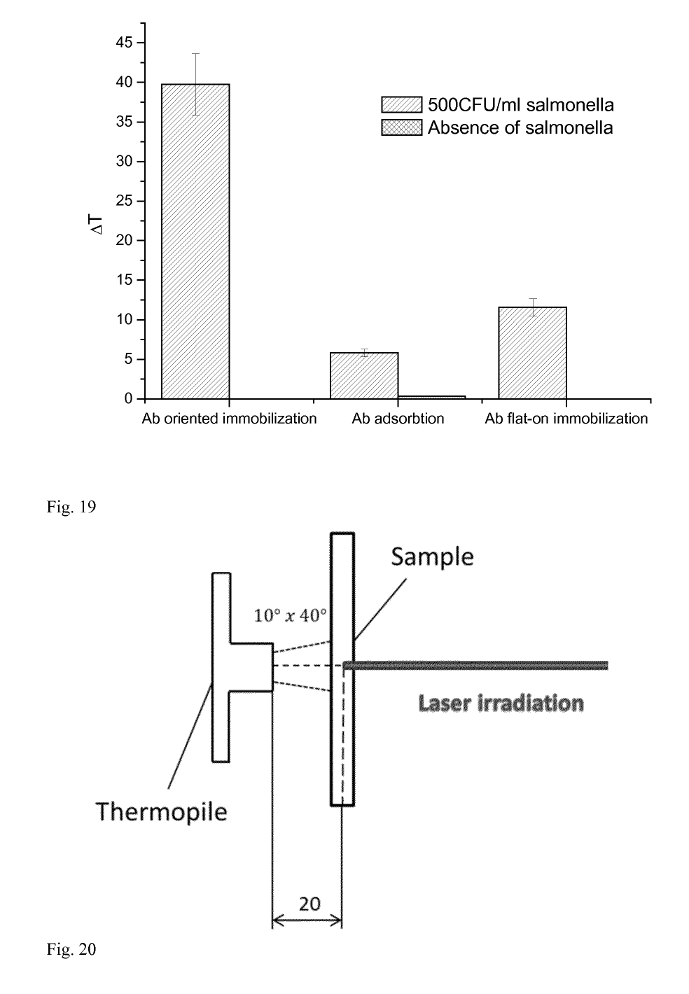

FIG. 19. Comparison between different surface functionalizations of the glass surface

FIG. 20. Disposal 1: Thermopile behind sample.

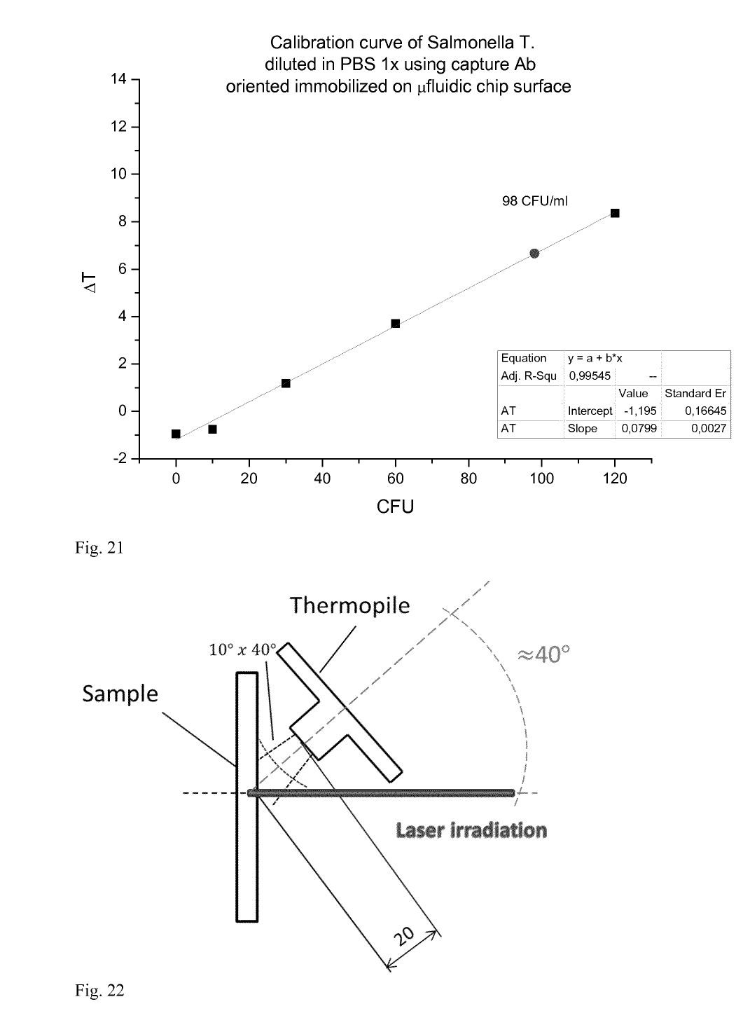

FIG. 21. Disposal 1: Calibration curve of Salmonella T.

FIG. 22. Disposal 2: Thermopile in front of sample.

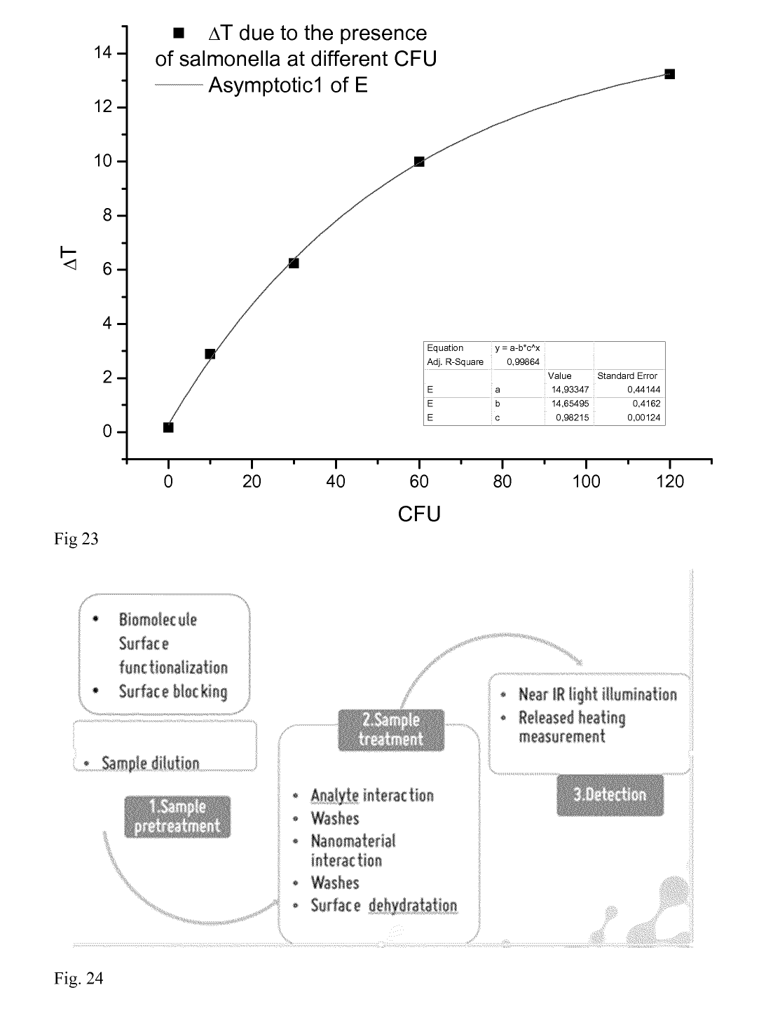

FIG. 23. Disposal 2: Calibration curve Salmonella T.

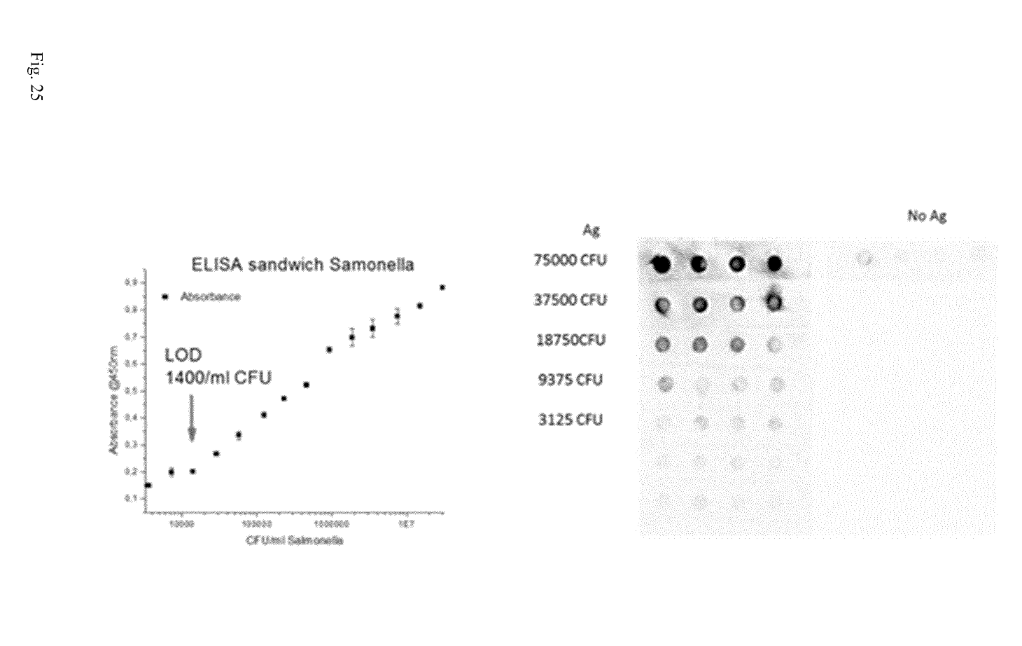

FIG. 24. Flowchart.

FIG. 25. Detection of Salmonella (Ag) at different CFU with ELISA and sandwich dot-blot. As negative control the dot-blot has been carried out in absence of salmonella (No Ag).

FIG. 26. General protocol implemented for the detection of salmonella using HEATSENS.

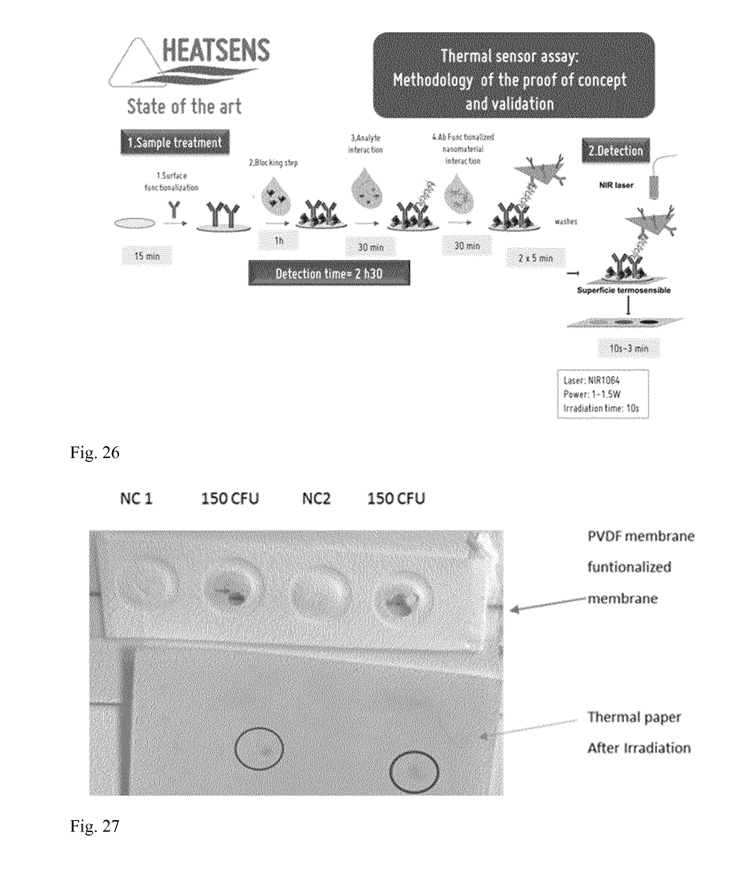

FIG. 27. HEATSENS detection of 150 CFU of salmonella in a 200 microliters sample using a visual method.

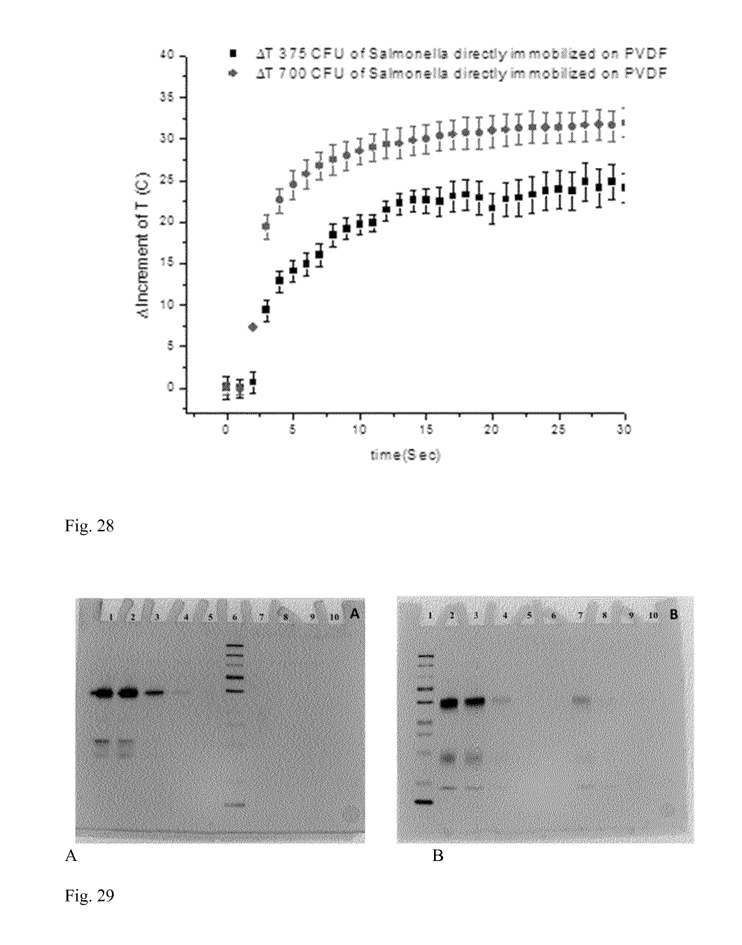

FIG. 28. Measurement of the increment of temperature due to the detection of Salmonella at different CFU directly adsorbed onto PVDF membrane. The sample was irradiated for 30 sec with a NIR beam at 0.4 W.

FIG. 29. A) SDS-PAGE gel of gold nanoparticles functionalized with NTA-Co2+ and Anti CD3: B) SDS-PAGE gel of gold nanoparticles functionalized with NTA-Cu2+ and Anti HRP. Lanes: (A) (1) Anti-CD3 35 μg/mL; (2) Supernatant AuNP-NTA-Co2+; (3) Supernatant after wash 1; (4) Supernatant after wash 2; (5) Supernatant after wash 3; (6).

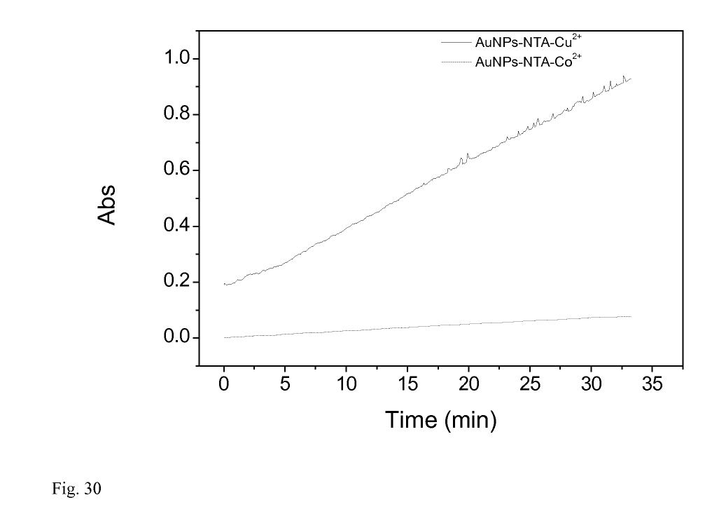

FIG. 30. Activity of gold nanoparticles functionalized with NTA-Cu2+(blue line) and NTA-Co2+ (red line) after incubation with anti-HRP and enzyme HRP. Conditions: 1 mM ABTS as electron donor and 1 mM H2O2 as electron acceptor in 50 mM sodium phosphate buffer, pH 6.0 at 25° C.

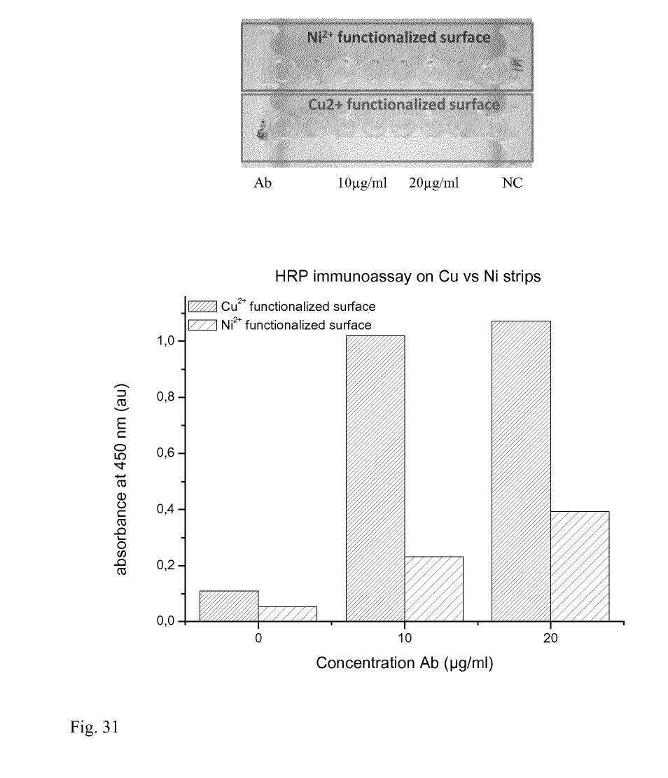

FIG. 31. Immunoassay for the detection of HRP on commercial strips activated with nickel and copper ions. A) activity of HRP on surface functionalized with Ni and Cu and antibodies anti HRP immobilized at 10 and 20 μg/ml; B) Absorbance at 450 nm relative to the TMB substrate after HRP activity.

FIG. 32. HEATSENS measurement of HRP immunoassay carried out using commercial strips activated with copper and nickel ion and functionalized with at 10 and 20 μg/ml of antibodies anti-HRP.

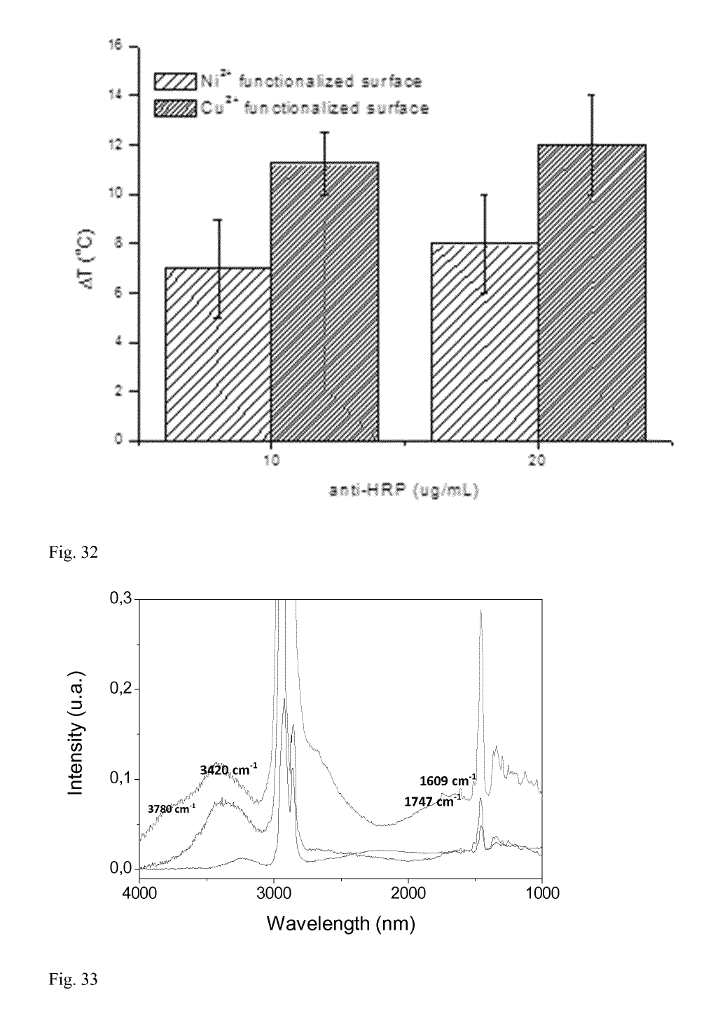

FIG. 33. FTIR spectra of modified COC samples with NTA-Cu2+ using our procedure (red line); NTA-Ni2+ using UV radiation (blue line). FTIR spectrum of untreated COC sample (black line).

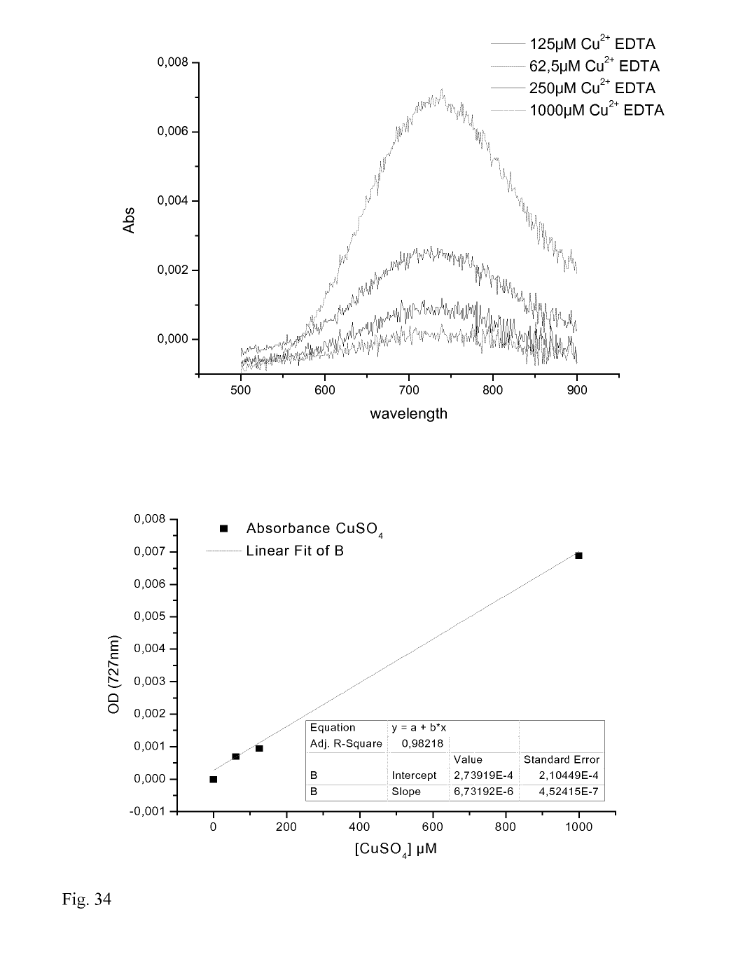

FIG. 34. UV-vis spectra of calibration points and the relative max of absorbance at 727 nm of CuSO4 in EDTA solution.

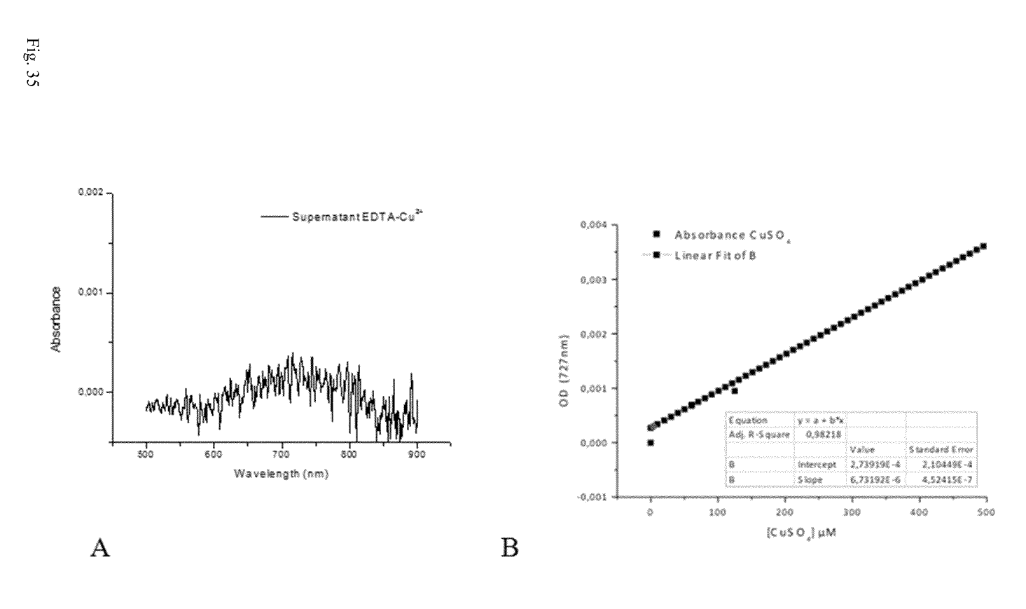

FIG. 35. A) UV-Vis spectrum of Cu-EDTA removed from microfluidic chip surface obtained after step incubation. B) extrapolation of Cu2+ concentration on surface analyzed.

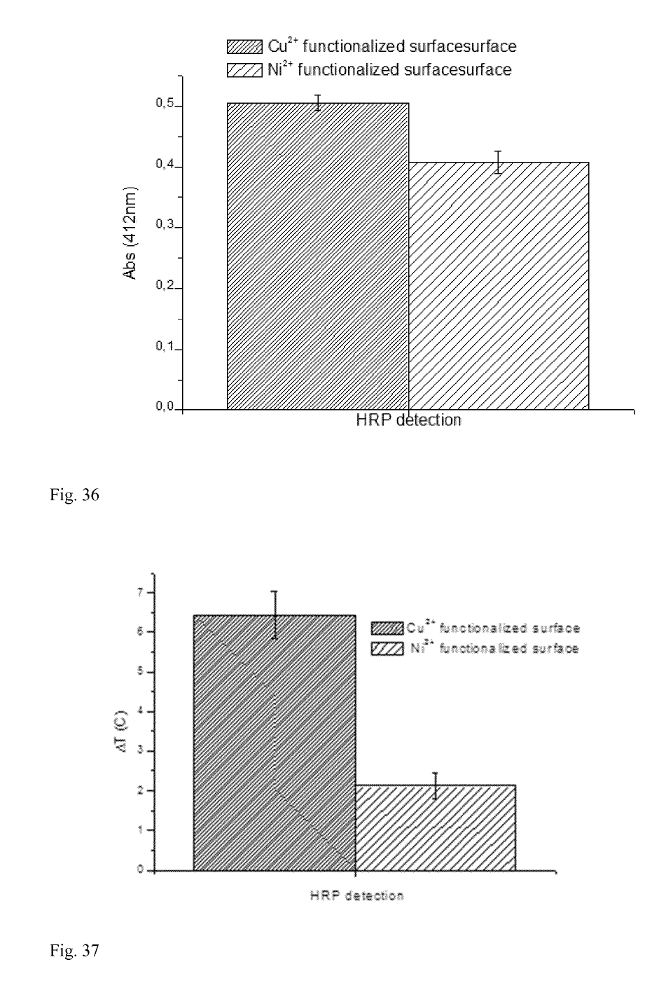

FIG. 36. Immunoassay for the detection of HRP on surfaces activated with nickel (comparative example) and copper ions (NIT). The activity of HRP on surface functionalized with Ni and Cu and antibodies anti HRP immobilized; Absorbance at 412 nm relative to the ABTS substrate after HRP activity.

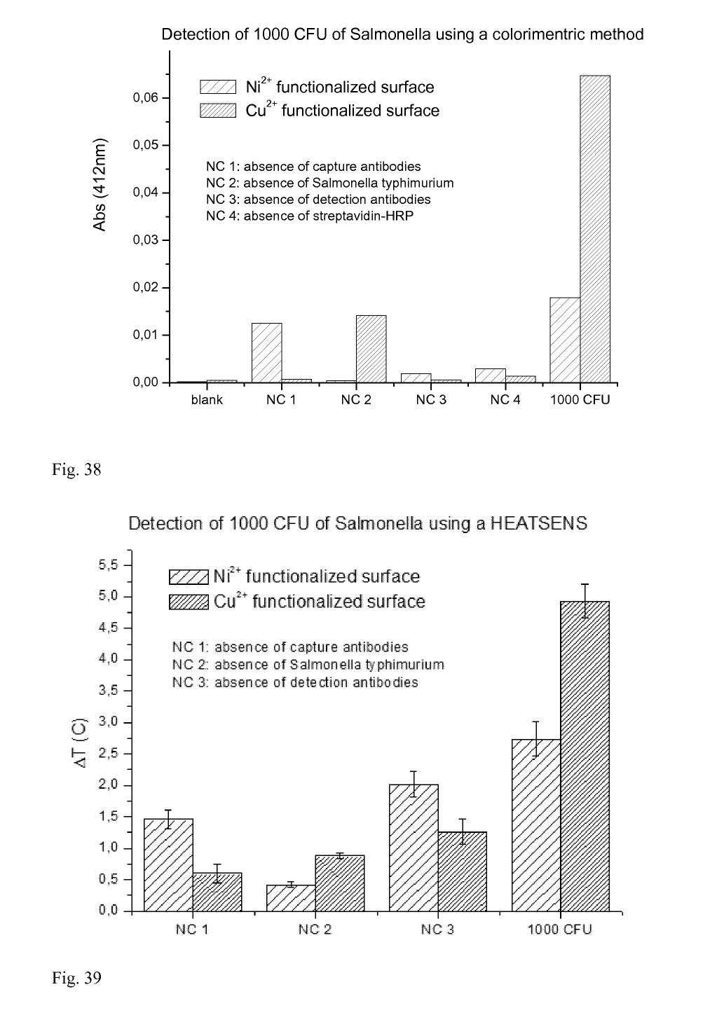

FIG. 37. Increment of temperature (ΔT) due to the presence of biotinylated HRP, captured by the oriented immobilized antibodies on the two metals chelated surfaces FIG. 38. Colorimetric Immunoassay for the detection of Salmonella T. on surfaces activated with nickel (comparative example) and copper ions (NIT). The adsorbance at 412 nm of the positice control is reported in comparison with negative controls, where NC1 refers to the assay in absence of the apture antibodies, NC2 refers to the annsay in absence of analyte Salmonella, NC3 refers to the assay in absence of the detection antibody and NC4 refers to the absence of strepavidin-HRP.

FIG. 39. HEATSENS Immunoassay for the detection of 1000 CFU Salmonella T. on surfaces activated with nickel (D4) and copper ions (NIT) assay: measurements of the increment of temperature (ΔT) relative to the presence of analyte onto differently functionalized surfaces. The adsorbance at 412 nm of the positice control is reported in comparison with negative controls, where NC1 refers to the assay in absence of the apture antibodies, NC2 refers to the annsay in absence of analyte Salmonella, NC3 refers to the assay in absence of the detection antibody.

### DETAILED DESCRIPTION OF THE INVENTION

### Definitions

For the purpose of the present invention, the following definitions are included below:
The term “comprising” it is meant including, but not limited to, whatever follows the word “comprising”. Thus, use of the term “comprising” indicates that the listed elements are required or mandatory, but that other elements are optional and may or may not be present.
By “consisting of” is meant including, and limited to, whatever follows the phrase “consisting of”. Thus, the phrase “consisting of” indicates that the listed elements are required or mandatory, and that no other elements may be present.
It is also noted that the term “kit” or “device” as used herein is not limited to any specific device and includes any device suitable for working the invention.
As used herein “Microfluidics” is the science that deals with the flow of liquids inside micrometer-size channels. In order to consider it microfluidics at least one dimension of the channel must be in the range of a micrometer or tens of micrometers. Microfluidics can be considered both as a science (study of the behaviour of fluids in micro-channels) and as a technology (manufacturing of microfluidics devices for a variety of applications as the one disclose herein for identifying and quantifying analytes).
As used herein “microfluidic chip or device” refers to a set of micro-channels etched or molded into a material (such as glass, silicon, a thermoplastic material or a polymer such as PDMS, for PolyDimethylSiloxane). The micro-channels forming the microfluidic chip are connected together in order to achieve the desired features (mix, pump, sort, control bio-chemical environment). This network of micro-channels trapped into the microfluidic chip is connected to the outside by inputs and outputs pierced through the chip, as an interface between the macro- and micro-world. It is through these holes that the liquids (or gases) are injected and removed from the microfluidic chip (through tubing, syringe adapters or even simple holes in the chip) with external active systems (pressure controller, push-syringe or peristatic pump) or passive ways (e.g. hydrostatic pressure). Preferably, as used herein “microfluidic chip or device” is understood as a chip or device especially suitable for carrying out immunoassays, such as sandwich inmmunoassays, for detecting analytes which comprises a support or substrate, wherein said support or substrate comprises at least one channel in the substrate, the channel comprising an inlet, an outlet, and a flow-path connecting the inlet and outlet, wherein the inlet and outlet together define a midplane; and a portion of the flowpath travels transversely across the midplane, wherein the portion of the flowpath that travels transversely across the midplane includes a recognition site or sensing area for detecting a target analyte.
As used herein the term “Heatsens methodology or technology” is understood as any methodology that uses the light to heat conversion properties of metal nanoparticles as a signal transduction system. The basis for using this system as a tag in biosensors is due to the presence of the surface plasmon absorption band. These absorption bands are produced when the frequency of the light striking the nanoparticle is in resonance with the collective oscillation frequency of the electrons in the particle conduction band, causing excitation. This phenomenon is known as “localized surface plasmon resonance” (LSPR). The position in the spectrum of the resonance band greatly depends on the particle shape, size and structure (hollow or solid), as well as on the dielectric medium where the particle is found. LSPR leads to high molar extinction coefficients (˜3×10¹¹ M⁻¹ cm⁻¹), with an efficiency equivalent to 10⁶ fluorophore molecules and a significant increase in the local electric field close to the nanoparticle. Metal nanoparticles such as gold, silver or copper nanoparticles have this surface plasmon resonance effect. When irradiated with a high intensity external light source with the suitable frequency, such as a laser, these particles are capable of releasing part of the absorbed energy in the form of heat, causing a localized temperature increase around their surface.
As used herein the term “metal nanoparticle” is understood as any mono- or polycrystalline cluster of metal atoms in any of their oxidation states, or any of their alloys, having all geometric dimensions between 1 and 1000 nm, preferably between 1 and 200 nm, measurable using standard electro-microscopy, with photonic properties. The metal nanoparticles disclosed herein can be symmetric or asymmetric, and have a variety of shapes such as rods, prisms, stars or nanocages. The metal particles disclose herein must have the capability to absorb light and generate heat in an efficient way. The term “efficient way” is well understood by the skilled person but, without being limited by this value, efficient way may be understood as 0.03 C/sec which is the value that results from the slope of the plot temperature vs irradiation time as measured by standard means. In a preferred embodiment of the invention, said metal atoms are noble metals. In a more preferred embodiment of the invention, said metal atoms are gold, silver or copper atoms. In an even more preferred embodiment of the invention, they are tubular or triangular gold or silver atoms.
As used herein the terms “carboxylic functional groups” or “epoxy functional groups” or “amine functional groups” or “thiol functional groups” or “azide functional groups” or “halide” or “maleimide functional groups” or “hydrazyde functional groups” or “aldehyde groups” or “alkynes groups”, are used herein as understood by the common general knowledge.
In the context of the present invention, external light source is understood as any electromagnetic radiation source with energy between 380 nm and 1100 nm, with the capacity to cause excitation of the LSPR band of metal particles based on gold, silver, copper or any of their alloys or oxidized states, preferably in the near infrared range (between 750 and 1100 nm) because energy absorption by the interfering biomolecules present in the sample which absorb in the visible range of the spectrum (hemoglobin, etc.) does not occur in that energy range.
In the context of the present invention, recognition molecule or capture biomolecule is understood as any molecule capable of specifically recognizing a specific analyte through any type of chemical or biological interaction.
In the context of the present invention, second recognition molecule or detection biomolecule is understood as any molecule capable of specifically recognizing a specific analyte through any type of chemical or biological interaction.
The molecules used as recognition elements in the biosensors of the present invention must have a sufficiently selective affinity for recognizing a specific analyte in the presence of other compounds, in addition to being stable over time and preserving their structure as well as their biological activity once immobilized on the support and on the surface of the nanoparticles. Antibodies, peptides, enzymes, proteins, polysaccharides, nucleic acids (DNAs), aptamers or peptide nucleic acids (PNAs) can be used as recognition molecules in the developed system.

### DESCRIPTION

The present invention provides a solution for offering a highly specific and sensitive method for the identification of a large variety of analytes, such as food pathogens as Salmonella, E. coli or Campylobacter, allergens such as Ara H 1 or other analytes such as collagen or albumin, in a rapid an efficient way.

For this purpose, the authors of the present invention combined the use of a number of functionalized surfaces with antibodies capable of detecting the target analyte by using the Heatsens technology (see definitions). Therefore, controlled heat generation in combination with functionalized surfaces with antibodies was chosen as the basis for the new generation of detection systems developed in the present invention. The phases of the protocol for the detection of the analyte used herein are summarized in the flowchart shown in FIG. 24, divided in tree main phases: sample pre-treatment, treatment and detection.

In order to implement this technology, we first tested the adequate coupling of antibodies specific against Salmonella typhymurium using the ELISA technique and a dot-blot assay format. As analyte, an attenuated Salmonella typhymurium from BacTRace (https://www.kpl.com/catalog/productdetail.cfm?catalog_ID=17&Category_ID=415&Product_ID=952) was used as model to implement and optimize the assay.

The detection of Salmonella using this standard methodology (ELISA) using enzymes as labels of the analyte presence, achieved a limit of detection (LOD) of 1.400 CFUs in the case of the ELISA assay and 3.125 CFU in the case of the detection with a dot-blot assay format, as shown in FIG. 25. Once demonstrated that the antibodies bounded to the target analyte (Salmonella typhymurium), several surfaces, where to carry-out the detection, were tested in combination with the Heatsens technology for the development of the sensing platform. In particular, the following surfaces were chosen: Nitrocellulose, PVDF, Cyclo Olefin Polymer (COP) and Patterned TiO2 film.

The above-mentioned surfaces were selected due to their different capacities for their functionalization with antibodies and for their thermal conductivity, reported herein below:
Nitrocellulose 0.12-0.21 W/(m K).
PVDF W/m-K 0.17-0.19.
Cyclo Olefin Polymer (COP) (microfluidic chip) 0.12-0.15 W/(m K).
Patterned TiO2 film 11.8 W/m·K.

As shown through-out the present invention, an ideal surface to be used as the detection surface has to: i) allow the use of functionalization methodologies to ensure an oriented binding, and ii) have a high thermal conductivity. Increasing the thermal conductivity of the detection support used for HEATSENS will improved the sensitivity of the immunodetection of the analyte, since the heat released by the metal nanoparticles interacting with the analyte, will be measured in a faster and more precise way from the thermal detector.

We herein below describe the functionalization of the different surfaces tested herein:
Nitrocellulose/PVDF: 15-25 μl per dot of the capture antibody at the proper concentration in the correct buffer (being careful of adding the drop in the center and near the nitrocellulose or PVDF) was deposited using a dot-blot system at a vacuum of 700 mbar and remained drying at 700 mbar vacuum for 10 minutes. After that, the antibody-functionalized membranes were washed two times adding 4 ml of washing solution (PBS buffer with 0.5% of BSA and 0.5% of Tween), and incubated at room temperature for 10 minutes with agitation before the solution was discarded. The membranes were then incubated with 5 ml of blocking solution (PBS buffer with 5% of BSA and 0.5% of Tween) for 60 min at 37° C. with agitation and washed two more times in previous mentioned conditions. After that, the nitrocellulose membrane was ready for the incubation with the analyte.
Patterned TiO2 film: 5 μg/ml of capture antibody was adsorbed and the surface was then blocked.
Microfluidic chips: made of cyclo olefin polymers and PMMA, were functionalized with 5 μg/ml of the capture antibody by physical adsorption onto the polymeric surface. In the same way also different CFUs of salmonella were directly adsorbed onto the chip surface in order to test not only a sandwich assay but also a direct immunoassay.

To perform the incubation with the analyte of the nitrocellulose or PVDF membranes and the patterned TiO2 film, they were incubated with 200 μl of the different concentrations of the analyte in a buffer (respectively buffer phosphate, peptone culture media, and real sample) for 30 min at 37° C. with agitation. The incubated supports were washed two times adding 400 μl of washing solution (PBS buffer with 0.5% of BSA and 0.5% of Tween), and were incubated at room temperature for 5 minutes with agitation. When this washing step was finished two additional washing steps were performed adding 400 μl of sodium phosphate buffer 10 mM pH 7, incubating the surfaces at room temperature for 5 minutes with agitation. The final step of the detection was the incubation of the support with 20 μg/ml of streptavidine@nanoprisms, diluted in blocking buffer (PBS buffer with 5% of BSA and 0.5% of Tween) for 30 min at 37 C. The surfaces were then dried for 15 minutes at 37° C. FIG. 26 illustrates the general scheme of the validated procedure to perform the immunoassay.

For each experiment we validated the specific interaction of the antibodies with the salmonella, introducing the following controls:
the absence of the capture antibody;
the absence of the analyte;
the absence of the biotynilated detection antibody;
the absence of streptavidine@Nanoprisms.

The detection of Salmonella was first made in a semi-quantitative way using a thermal paper coupled to the functionalized membrane/support and displayed as the burning of the thermal paper. The support used was PVDF functionalized with capture antibody for testing the capture and of course detection, of the different dilutions of salmonella, in a range between 150 CFUs and 6.000 CFUs in 200 microliter samples. The illumination of the membrane, after incubation with the nanoprisms functionalized with the detection antibody, achieved the visual detection of 150 CFUs in a 200 microliter sample of Salmonella, detection shown in FIG. 27.

However, the above visual method did not achieve a satisfactory detection limit for use in food contaminated samples wherein the pathogen is scarcely present in just a few CFUs/ml such as in an amount <90 CFUs/ml.

In order to solve this problem, the authors of the present invention try to use a quantitative detection using commercial thermopiles. In this sense, it is noted that the heat released by nanoprisms upon IR illumination can be measured by using an IR thermopile, such as a MIX90620 from Melexis. This thermopile is suitable to detect thermal radiation and measure temperatures without making contact with the sample.

The MIX90620 thermopile contains 64 IR pixels with dedicated low noise chopper stabilized amplifier and fast ADC integrated. A PTAT (Proportional to Absolute Temperature) sensor is integrated to measure the ambient temperature of the chip. It requires a single 3V supply (+0.6V) although the device is calibrated and performs best at VDD=2.6V. The MLX90620 is factory calibrated in wide temperature ranges: −40 . . . 85° C. for the ambient temperature sensor −50 . . . 300° C. for the sample temperature. Each pixel of the array measures the average temperature of all objects in its own Field Of View (called nFOV).

For the quantitative detection, salmonella was directly immobilized onto a PVDF support at different CFUs dilutions, in the range within 375 to 6.000 CFUs, in 200 microliter samples, in particular a dilution containing 375 CFUs and a dilution containing 700 CFUs were used. Detection was performed in a quantitative way by measuring the increment of temperature generated by the presence of nanoprisms interacting with the analyte, as shown in FIG. 28.

As a negative control, we measured the temperature increased of those membranes that followed the same protocol of detection but were not incubated with salmonella. As expected, in the absence of salmonella, the nanoprisms did not interact with the membrane, as we did not observe an increase of the increment of temperature of this control.

In the presence of the salmonella, previously diluted in buffer phosphate and directly adsorbed onto surface, the increment of temperature of 375 CFU was of approx. 19° C., meanwhile 700 CFU of salmonella, generated an increment of approx. 27° C. However, as with the visual method, we did not achieve a satisfactory detection limit for use in food contaminated samples wherein the pathogen is scarcely present in just a few CFUs/ml such as in an amount <90 CFUs/ml.

To solve this problem, we then tried using supports other than PVDF and nitrocellulose, such as TiO2 patterned supports. Yet, as with the visual detection method and the quantitative detection methods shown so far, a satisfactory detection limit was again not achieved in a reliable way.

In order to solve this problem, we then tried combining the microfluidic technology with the Heatsens technology in order to carry out a series of immunoassays capable of detecting a target analyte with a satisfactory detection limit in a reliable way. For this purpose, the unmodified fabricated microfluidic chip illustrated in the materials and methods of the examples was used for testing the direct immobilization of two dilutions of salmonella. For this purpose, 10 μl of 60000 CFU/ml and 20000 CFU/ml (600 and 200 CFU in total on the surface, respectively) of Salmonella T. were adsorbed on the detection surface. After the direct immobilization of the pathogen, the surface was blocked with BSA and allow to react with biotinylated detection antibodies. Finally, they were washed and further reacted with streptavidin-AuNanoprisms solution.

In FIG. 2, we show the increment of temperature measured upon NIR irradiation of the surface due to the presence of the Salmonella after its recognition by biotinylated detection antibodies and further interaction with streptavidine-Nanoprisms. In absence of biotinylated detection antibody (NC2) there is an insignificant increment in temperature as in absence of strepavidin@AuNPrism (NC3). The increment of temperature was proportional to the amount of salmonella's CFUs. These results indicate the suitability of this material for the fabrication of the microfluidic chip and its application for HEATSENS. Moreover, the results envisaged the possibility of immobilizing salmonella at different CFU dilutions directly onto a microfluidic chip and build a calibration curve.

In view of these results, we then used 10 μl of different concentrations (CFU/ml) of salmonella T, in a range between 0 and 240000 CFU/ml. These were directly adsorbed onto the microfluidic chip and detected with biotinylated antibodies anti-salmonella to measure the increment of temperature due to the presence of different concentrations of salmonella. Then the strepavidine@AuNprism interacted with the antibodies and every single sensing area was irradiated with an IR laser. The temperature of each chamber was measured, and the increment of temperature calculated. FIG. 3 displays the calculated increment of temperature in function of the amount of salmonella's CFU/ml.

The increase of temperature measured was due to the increased amount of CFUs directly adsorbed onto the surface of microfluidic chip.

Once shown that the microfluidic chip was suitable to be applied to the HEATSENS technology, we performed a sandwich type immunoassay for the detection of the selected pathogen by using a microfluidic chip. For this purpose, each micro-chamber of the microchip was functionalized with capture antibodies anti-salmonella by direct adsorption of (5 μL) 5 μg/ml of capture antibodies anti-salmonella onto the surface. Then, the salmonella's capture event was carried out in fluidic mode, as well as the detection and the interaction with the streptavidin-AuNprism, injecting 1 ml of sample, in each channel. The assay was carried out with 2 different concentrations of salmonella's CFU/ml, 200000 CFU/ml and 240000 CFU/ml diluted in buffer phosphate, respectively. FIG. 4 describes the trend of the increments of temperature due to the presence of Salmonella T. The trend of the calibration curve was not linear, indicating a saturation of the signal due to the presence of high amount of nanoprisms interacting with the analyte. The detection of the two unknown concentrations of salmonella was calculated from the exponential equation, where the values concur with the curve with an adj. R-Square equal to 0.98843.

Once shown the effectiveness of an immunoassay in a sandwich format, we tried to improve the limit of detection of salmonella t., by decreasing the concentration of the pathogen in doped buffer. In this sense, 1500 CFU/ml of salmonella T. in PBS 1× was the first lower concentration detected in the first trial. For this purpose, a 1 ml sample was injected in the channel with a flow of 200 μl/ml. After injecting the sample, the channel was washed with washing buffer (BSA 0.5% in PBS1×, 0.1% tween), using a flow of 300 μl/min for 4 min. Then the detection antibodies were allow to interact with its antigen using a 200 μl/ml for 2 minutes. The channel was rinsed with washing buffer (BSA 0.5% in PBS1×, 0.1% tween), using a flow of 300 μl/min for 4 min. The streptavidin@AuNPr were injected into the channel. The flow was 200 μl/ml for 2 minutes. The channel was rinsed with washing buffer (BSA 0.5% in PBS1×, 0.1% tween), using a flow of 300 μl/mim for 4 min and dried.

FIG. 5 illustrates the increment of temperature of 1500 CFU/ml of salmonella with respect to the negative controls. The increment of temperature of the micro-chambers in the presence of salmonella was higher that the temperature increments of the controls, respectively in absence of salmonella (NC1), absence of detection antibodies (NC2), and absence of strepavidine-AuNPrism (NC3). The increment of temperature due to the presence of salmonella was higher than all negative controls, even though different from the expected value: the positive values of increment of temperature of the negative controls indicated non-specific interactions between the reagents within the immunoassay. The non-specific interactions can be associated to an uncompleted functionalization and blocking of the surface or to an inappropriate flow rate during the immunoassay. In this way, by keeping constant the surface antibody functionalization and modifying the flow rate during the immunoassay, it was possible to improve the limit of detection of salmonella and the signal due to the background, as shown in FIG. 6.

The same experiment was carried out using a real food sample, 25 g of chicken meat in 225 ml of peptone pre-enrichment culture media, doped with salmonella at different CFUs. The capture antibodies were adsorbed onto the microfluidic chip, and the surface blocked with 5% BSA in PBS1×-01% Tween, using a flow rate of 15001/min.

Then, the washing was carried out using a flow rate of 250 μl/min, by using a washing buffer.

The capture of salmonella in a 1 ml of real sample, as well as the detection with biotinylated detection antibodies, and the interaction with streptavidin@nanoprisms was performed by using a flowing at a flow rate of 15 μl/min.

The results of the immune analysis carried out in the microfluidic chip are shown in FIG. 7. After building the calibration curve, measuring the increment of temperature due to the known different concentrations of salmonella, the unknown concentration of pathogen in the real sample was determined from the calibration curve (FIG. 8).

The higher increment of temperature of the samples doped with salmonella, clearly indicates that HEATSENS in combination with the microfluidic technology is suitable for the ultrasensitive detection of few CFUs of bacteria in complex matrices such as the 25 g of chicken meat in 225 ml of peptone.

The increment of temperature due to the presence of salmonella in a real sample is slightly different from the one in buffer phosphate, because of presence of high amount of meat proteins which affect the specific interaction of the bacteria with the antibodies.

Once shown that the combination of HEATSENS with the microfluidic technology is suitable for the ultrasensitive detection of few CFUs of an analyte, we tried to improve this technology by modifying the microfluidic chip surface with carboxylic end groups used to immobilize covalently capture antibodies by the formation of stable amide bonds with their primary amines via EDC/sulfo-NHS reaction.

In this sense, the surface of each micro-chamber, previously activated with 10 mM EDC and 20 mM sulfo-NHS, was functionalized with 20 μl of 5 μg/ml of capture antibodies. After the covalent immobilization of the capture antibodies and the blocking of surface with BSA 5% in PBS1×/0.1% Tween for 1 hour at 37 C, the chip was connected to the peristaltic pump and washed with washing buffer using a flow rate of 300 μl/min for 4 minutes. 1 ml of 30 CFU/ml of Salmonella T., were allow to flow inside the microfluidic channel for 1 minute at a flow rate of 150 μl/min, then the channel was washed with a buffer solution using a flow rate of 300 μl/min for 4 minutes. 400 μl of biotinylated detection antibodies were then flowed inside the channel.

The results depicted in FIG. 9 show that the temperature increment in the sample doped with 30 CFU/ml of Salmonella is higher in comparison with those of different controls. In this type of immobilization, the antibody adopts a predominantly “flat-on” orientation with the Fc and two Fab fragments lying flat on the surface.

We then tried an oriented immobilization of the antibodies through metal-chelation. Immobilization was accomplished through the metal-chelation to histidine-rich metal binding site in the heavy chain (Fc) of the antibody or to poly-His-tag sequence fused in proteins. Since the metal binding site is either in the C- or N-terminus, antibodies and His-tagged proteins bound in this fashion to the surface are oriented with the combining site directed away from the surface thus allowing maximal antigen binding or a favourable protein orientation. Furthermore, oriented immobilization through metal-chelation also results in a stable antibody immobilization since binding constants for metal-chelation immobilization are very high due to the combination of the chelate effect of histidine binding, and target binding of multiple metal-chelate groups. Dissociation constants are estimated to be between 10⁻⁷ to 10⁻¹³ M⁻¹. For many applications, this provides binding strengths comparable to antigen-antibody interaction. On the other side, experimental conditions of antibody attachment for oriented immobilization of antibodies through metal-chelation are milder than those employed for covalent oriented immobilization procedure. As an advantage, the antibody binding to the chelate could be also modulated as convenience to be reversible or irreversible. In addition, it is also more versatile since it can be also employed for immobilization of his-tagged recombinant proteins.

In order to achieved an oriented immobilization of the capture antibodies, the microfluidic chamber chips were functionalized with metal-chelate complexes in a stepwise modification of their surface. Firstly, the surfaces were functionalized with aryl amine compounds containing carboxylic groups such as for example 3-(4-Aminophenyl)propionic acid, 3-Aminophenylacetic acid, 4-Aminophenylacetic acid or 4-(4-Nitrophenyl)butyric acid. For this specific example we used PhBut, even though for the immobilization of different biomolecules, it would be more appropriate the use of aryl amine compounds carrying different lengths of n-alkyl carboxylic acids in a range between 2 and 16 carbons.

Carboxylic groups introduced by covalent grafting of the aryl radical of diazotated PhBut (Scheme II) were activated by esterification with SNHS catalyzed by EDC to facilitate the covalent linkage of the ANTA-M(II) (Cu2+, Ni2+, Co2+) complex (Scheme III) through the free amino groups. Then, they were incubated with 20 μl of 5 μg/ml of capture antibodies. The resulting NTA-M(II) complex termination contains two free coordination sites occupied by water molecules to be replaced by histidine residues of capture antibodies giving rise to their oriented immobilization. Later, the chip was connected to the peristaltic pump and washed with washing buffer using a flow rate of 300 μl/min for 4 minutes. 1 ml of 30 CFU/ml of salmonella T., was allow to flow inside the microfluidic channel for 1 minute at a flow rate of 150 μl/min, then the channel was washed with buffer using a flow rate of 300 μl/min for 4 minutes. 400 μl of biotinylated detection antibodies was then flowed inside the channel.

FIG. 10 illustrates the detection of salmonella on a microfluidic chip functionalized with capture antibodies in an oriented manner.

Interestingly, the temperature increment due to the presence of Salmonella for this type of immobilization was higher than those obtained for the respective controls and even higher than those obtained in previous results for direct adsorption and covalent immobilization. In this sense, a comparative study between the different immobilization methods was carried out. The comparison of the different strategies of antibody surface functionalization is displayed in FIG. 11, where it is shown the increment of temperature due to the detected Salmonella in comparison with the generated background signal, for each of the surface functionalization strategies shown in this example.

FIG. 11 shows that the oriented immobilization of capture antibodies through metal-chelation provides the best results not only by providing the higher temperature increment due to the presence of salmonella but also by providing the lower signal generated by non-specific interactions (background). These results indicate that a correct functionalization strategy of the surface of the chip is crucial in order to obtain an optimal antibody attachment in a favorable orientation, while avoiding non-specific adsorptions of HEATSENS labels (such as gold nanoprisms). It is also noteworthy, that this method shows advantages over covalent oriented immobilization. Although, both methodologies have the advantage of obtaining an oriented antibody attachment for binding, in the case of metal-chelation immobilization the antibody is placed oriented perpendicular to the surface “end-on” orientation in contrast to the covalent immobilization where the antibody adopts a predominantly “flat-on” orientation, with the Fc and two Fab fragments lying flat on the surface.

The advantageous antibody oriented immobilization shown in example 6, was then tested for the detection of salmonella in a real sample. The result is reported in FIG. 12, which illustrates the increment of temperature due to the salmonella in a real sample doped with a known number of Salmonella CFUs, in comparison with the signals generated by the negative controls.

The temperature increment, due to the presence of salmonella in the real sample, on an oriented antibody immobilized microfluidic chip surface, was also higher than those obtained for the respective controls. After building the calibration curve, the measurement of the increment of temperature due to the known different concentrations of salmonella and to the unknown concentration of pathogen in the real sample was determined, as reported in the FIG. 13. The increment of temperature due to the presence of the theoretical number of CFU/ml used to dope the real sample, agrees with the number of CFUs of the calibration curve.

Therefore, the microfluidic technology was thus selected as the best approach for the fabrication of a preferably disposable cartridge required to perform a HEATSENS protocol for analyte detection. Moreover, the microfluidic technology in combination with an oriented configuration of the capture biomolecules (such as antibodies) has been shown herein as an excellent approach for the fabrication of a preferably disposable cartridge required to perform a HEATSENS protocol for analyte detection.

Lastly, it is important to note that as clearly illustrated in examples 8, 9 and 10, the combination of the microfluidic technology and the Heatsens technology is suitable for the detection and quantification of a large variety of analytes such as, but not limited to, microorganisms, additives, drugs, pathogenic microorganisms such as a food pathogens, food components, environmental contaminants, pesticides, nucleotides, biomarkers such as medical biomarkers or toxic compounds etc. Therefore, the sensor systems described herein are not limited to any specific analyte.

Use of the Microfluidic Technology in Combination with the Heatsense Technology

Disclosed herein, in various exemplary embodiments, we show that microfluidic chips are especially suitable for use in a number of immunoassays for detecting an analyte as a result of the heat generated by metal nanoparticles when they are irradiated with an external light source.

These devices are useful for detecting the presence of one or more target analytes in one or more sample fluids. Methods and processes of making and using such devices are also disclosed in the examples.

Therefore, a first aspect of the invention refers to the in vitro use of a microfluidic kit or device comprising a support or substrate, wherein said support or substrate comprises at least one channel in the substrate, the channel comprising an inlet, an outlet, and a flow-path connecting the inlet and outlet, wherein the inlet and outlet together define a midplane; and a portion of the flowpath travels transversely across the midplane, wherein the portion of the flowpath that travels transversely across the midplane includes a recognition site or sensing area for detecting a target analyte;

for detecting an analyte as a result of the heat generated by metal nanoparticles when they are irradiated with an external light source.

In a preferred embodiment of the first aspect of the invention, the portion of the flowpath travels transversely across the midplane multiple times. In another preferred embodiment of the first aspect of the invention, the portion of the flowpath may travel substantially perpendicularly across the midplane. In another preferred embodiment of the first aspect of the invention, the portion of the flowpath may travel continuously towards the outlet from the inlet. In another preferred embodiment of the first aspect of the invention, the device has a plurality of channels. In another preferred embodiment of the first aspect of the invention, the device has a plurality of micro-chambers with recognition sites in each one or more channels. In yet another preferred embodiment of the first aspect of the invention, the inlet of each channel is connected to a common loading channel. In still another preferred embodiment of the first aspect of the invention, the device comprises the characteristics of the microchip or device described in the materials and methods section of the examples.

In addition, it is noted that the substrate or surface of the device of the first aspect of the invention, may be made from a variety of materials such as thermoplastic materials, silicon, metals, or carbon. Preferably, it may be made by poly(methyl methacrylate), polystyrene, poly(dimethylsiloxane), polyethylene terephthalate, polyethylene, polypropylene, polylactic acid, poly(D,L-lactide-co-glycolide), polycarbonate, cyclic olefin copolymers, silicon, glass etc.

As illustrated in the examples (see examples 6 to 9), functioanalizing the sensing area of the microchip's surface improves the characteristics of the sensor by providing a covalent or oriented configuration of the capture biomolecules.

Thus, in another preferred embodiment of the first aspect of the invention or of any of its preferred embodiments, the portion of the flowpath that travels transversely across the midplane that includes a recognition site or sensing area is functionalized with one or more carboxylic functional groups or epoxy functional groups or amine functional groups or thiol functional groups or azide functional groups or halides or maleimide functional groups or hydrazyde functional groups or aldehydes or alkynes groups.

Different manners of functionalizing these types of surfaces with the above mentioned functional groups are shown in the examples. Anyhow, in general, if the support or substrate is made of:
a. a thermoplastic material such as a co-olephin polymer, functionalization is carry out by using a diazonium aryl compound containing one or more carboxylic groups or epoxy groups or amine groups or thiol groups or azide groups or halides or maleimide functional groups or hydrazyde functional groups or aldehydes or alkynes groups;
b. silicon material such as polydimethylsiloxane (PDMS) or glass, functionalization is carry out through self-assembly with organo-functional alkoxysilane molecules carrying one or more carboxylic groups or epoxy groups or amine groups or thiol groups or azide groups or halides or maleimide functional groups or hydrazyde functional groups or aldehydes or alkynes groups;
c. a metal such as iron, cobalt, nickel, platinum, palladium, zinc, silver, copper or gold, functionalization is carry out through self-assembly with molecules capable of interacting with the metal, such as thiol groups in the case of gold and silver, carrying one or more carboxylic groups or epoxy groups or amine groups or thiol groups or azide groups or halides or maleimide functional groups or hydrazyde functional groups or aldehydes or alkynes groups;
d. a carbon material such as graphene, functionalization is carry out as established in step a) above or through an oxidation to generate aldehydes and carboxylic functional groups or through hydrophobic binding of functionalized polymers having one or more carboxylic groups or epoxy groups or amine groups or thiol groups or azide groups or halides or maleimide functional groups or hydrazyde functional groups or aldehydes or alkynes groups.

Preferably, any of the above surfaces is functionalized with carboxylic functional groups. More preferably, the support or the microfluidic chip or device is made of a thermoplastic material and the diazonium aryl compound is represented by formula I or II below:

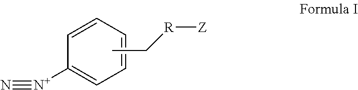

wherein R is an alkyl group having from 1 to 15 carbon atoms or an ethylene group; and Z is a carboxylic group, an epoxy group, an amine group, a thiol group, an azide group, a halide, a maleimide functional group, a hydrazyde functional group, an aldehyde group or an alkyne group, preferably a carboxylic or epoxy group; or

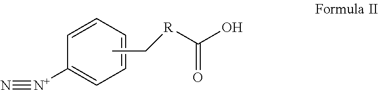

wherein R is an alkyl group having from 1 to 15 carbon atoms.

Preferably the diazonium component of formula I or II above is place or sited in the para or meta position with respect to the alkyl or ethylene component of any of these formulae. Examples of aryl amine compounds suitable for producing the diazonium aryl compound of any of formula I or II above are: 3-(4-Aminophenyl)propionic acid, 3-Aminophenylacetic acid, 4-Aminophenylacetic acid, 4-(4-Nitrophenyl)butyric acid or 4-(4-Aminophenyl)butyric acid (see examples).

In a further preferred embodiment of the first aspect of the invention, the surface of the microchip or device is further modified or functionalized with a chelating agent preferably selected from the list consisting of: Nα,Nα-Bis(carboxymethyl)-L-lysine hydrate (ANTA) metal (II) salt, nitrilotriacetic acid (NTA) metal (II) salt, iminodiacetic acid (IDA) metal (II) salt, Ethylenediaminetetraacetic acid (EDTA) metal (II) salt, diethylenetriaminepentaacetic acid (DTPA) metal (II) salt, wherein metal (II) salt is understood as a salt of a divalent metal such as Cu2+, Ni2+ or Co2+. This is accomplished by direct reaction of the chelating agent with any of the activated functional groups referred to above (with the exception of those groups like the epoxy groups that do not need to be activated to directly react with the chelating agent), wherein:
carboxylic groups can be activated via EDC/SNHS-mediated amidation (Scheme III);
amine groups can be activated with carbonyl groups;
thiol groups can be activated by forming sulfhydryl-reactive crosslinkers, wherein sulfhydryls can be selected from maleimides, haloacetyls or pyridyl disulfides;
alkyne or azide groups can be activated through CLICK chemistry;
aldehyde groups can be activated via the shift base formation.

Preferably, the support is made of a thermoplastic material and the aryl amine compounds contain carboxylic groups activated via esterification with N-Hydroxysulfosuccinimide salt (Sulfo-NHS) catalyze by (N-(3-Dimethylaminopropyl)-N′-ethylcarbodiimide hydrochloride (EDC).

More preferably, the support is made of a thermoplastic material, the aryl amine compounds contain carboxylic groups activated via esterification with N-Hydroxysulfosuccinimide salt (Sulfo-NHS) catalyze by (N-(3-Dimethylaminopropyl)-N′-ethylcarbodiimide hydrochloride (EDC), and the chelating agent is Nα,Nα-Bis(carboxymethyl)-L-lysine hydrate (ANTA) metal (II) salt, wherein metal (II) salt is understood as a salt of a divalent metal such as Cu2+, Ni2+ or Co2+.

As illustrated in the examples, activation of the microchip's surface with a chelating agent such as the ANTA metal (II) salt is especially advantageous to achieve an oriented configuration of the antibody resulting in an improved sensing platform.

In a further preferred embodiment of the first aspect of the invention or of any of its preferred embodiments, the portion of the flowpath that travels transversely across the midplane that includes a recognition site or sensing area may comprise:
a. a recognition molecule capable of recognizing the target analyte immobilized onto the recognition site or sensing area; or
b. an analyte immobilized onto the recognition site or sensing area.

Preferably, said recognition molecule can be selected from, but not limited to, the list consisting of: peptides, polysaccharides, toxins, protein receptors, lectins, enzymes, antibodies, antibody fragments, recombinant antibodies, antibody dendrimer complexes, nucleic acids, (DNA, RNAs), peptide nucleic acids (PNAs), molecular imprints. Preferably, said recognition molecule is an antibody, a fragment thereof or a recombinant antibody.

In a second aspect of the invention, the kit or device of the first aspect of the invention or of any of its preferred embodiments, may further comprise at least one of the following elements:
a. An external light source suitable for use in the Heatsens technology such as a laser or a LED light;
b. A second recognition molecule capable of recognizing the target analyte; or
c. A metal nanoparticle with photonic properties; and
d. Optionally a device capable of detecting the heat generated by the metal nanoparticles when they are irradiated with the external light source.

In a third aspect of the invention, the kit or device of the first aspect of the invention or of any of its preferred embodiments, may further comprise at least one of the following elements:
a. An external light source suitable for use in the Heatsens technology such as a laser or a LED light; or
b. A metal nanoparticle with photonic properties functionalized with a second recognition molecule capable of recognizing the target analyte; and
c. Optionally a device capable of detecting the heat generated by the metal nanoparticles when they are irradiated with the external light source.

In a fourth aspect of the invention, the kit or device of the first aspect of the invention or of any of its preferred embodiments, may further comprise at least one of the following elements:
a. An external light source suitable for use in the Heatsens technology such as a laser or a LED light;
b. A second recognition molecule (detection biomolecule) capable of recognizing the target analyte, optionally bound to a label molecule; or
c. Metal nanoparticles with photonic properties functionalized with biomolecules specifically recognizing the detection biomolecule or the label with which the detection biomolecule is modified; and
d. Optionally a device capable of detecting the heat generated by the metal nanoparticles when they are irradiated with the external light source.

Preferably the kit or a device of any of the second to fourth aspects of the invention, further comprises a device capable of detecting the heat generated by the metal nanoparticles when they are irradiated with the external light source selected from the list consisting of infrared cameras or thermopiles.

A fifth aspect of the invention refers to the use of the device according to any of the precedent aspects of the invention, wherein the analyte is a microorganism, additive, drug, a pathogenic microorganism such as a food pathogen, a food component, an environmental contaminant, a pesticide, a nucleotide, a biomarker such as a medical biomarker or a toxic compound. Preferably, the target analyte is selected from the list consisting of Salmonella, Campylobacter, collagen, albumin and Ara H1.

Microfluidic Device or Chip Having a Sensing Area Functionalized for an Antibody Oriented Immobilization.

As established in examples 6 to 9 by functionalizing the sensing area of a microchip or device so that a capture biomolecule such as an antibody has an oriented configuration provides clear advantages for the detection of an analyte in a sensor system combining the microchip technology with the Heatsens technology.

Thus, a sixth aspect of the invention refers to a kit or device comprising a support or substrate, wherein said support or substrate comprises at least one channel in the substrate, the channel comprising an inlet, an outlet, and a flow-path connecting the inlet and outlet, wherein the inlet and outlet together define a midplane; and a portion of the flowpath travels transversely across the midplane, wherein the portion of the flowpath that travels transversely across the midplane includes a recognition site or sensing area for detecting a target analyte;

wherein the portion of the flowpath that travels transversely across the midplane that includes a recognition site or sensing area is functionalized with a chelating agent.

In a preferred embodiment of the sixth aspect of the invention, the chelating agent is preferably selected from the list consisting of: Nα,Nα-Bis(carboxymethyl)-L-lysine hydrate (ANTA) metal (II) salt, nitrilotriacetic acid (NTA) metal (II) salt, iminodiacetic acid (IDA) metal (II) salt, Ethylenediaminetetraacetic acid (EDTA) metal (II) salt, diethylenetriaminepentaacetic acid (DTPA) metal (II) salt, wherein metal (II) salt is understood as a salt of a divalent metal such as Cu2+, Ni2+ or Co2+, preferably Cu2+. Preferably the chelating agent is selected from the list consisting of: Nα,Nα-Bis(carboxymethyl)-L-lysine hydrate (ANTA) metal (II) salt or nitrilotriacetic acid (NTA) metal (II) salt, wherein metal (II) salt is understood as a salt of a divalent metal such as Cu2+, Ni2+ or Co2+, preferably Cu2+. This is accomplished by direct reaction of the chelating agent with an activated functional group, wherein:
carboxylic groups can be activated via EDC/SNHS-mediated amidation (Scheme III);
amine groups can be activated with carbonyl groups;
thiol groups can be activated by forming sulfhydryl-reactive crosslinkers, wherein sulfhydryls can be selected from maleimides, haloacetyls or pyridyl disulfides;
alkyne or azide groups can be activated through CLICK chemistry;
aldehyde groups can be activated via the shift base formation.

Preferably, the support is made of a thermoplastic material and the aryl amine compounds contain carboxylic groups activated via esterification with N-Hydroxysulfosuccinimide salt (Sulfo-NHS) catalyze by (N-(3-Dimethylaminopropyl)-N′-ethylcarbodiimide hydrochloride (EDC).

More preferably, the support is made of a thermoplastic material, the aryl amine compounds contain carboxylic groups activated via esterification with N-Hydroxysulfosuccinimide salt (Sulfo-NHS) catalyze by (N-(3-Dimethylaminopropyl)-N′-ethylcarbodiimide hydrochloride (EDC), and the chelating agent is selected from the list consisting of: Nα,Nα-Bis(carboxymethyl)-L-lysine hydrate (ANTA) metal (II) salt or nitrilotriacetic acid (NTA) metal (II) salt, wherein metal (II) salt is understood as a salt of a divalent metal such as Cu2+, Ni2+ or Co2+, preferably Cu2+.

In a further preferred embodiment of the sixth aspect of the invention or of any of its preferred embodiments, the portion of the flowpath that travels transversely across the midplane that includes a recognition site or sensing area may comprise:
a. a recognition molecule capable of recognizing a target analyte immobilized onto the recognition site or sensing area; or
b. a target analyte immobilized onto the recognition site or sensing area.

Preferably, said recognition molecule can be selected from, but not limited to, the list consisting of: peptides, polysaccharides, toxins, protein receptors, lectins, enzymes, antibodies, antibody fragments, recombinant antibodies, antibody dendrimer complexes, nucleic acids, (DNA, RNAs), peptide nucleic acids (PNAs), molecular imprints. Preferably, said recognition molecule is an antibody, a fragment thereof or a recombinant antibody.

In a seventh aspect of the invention, the kit or device of the sixth aspect of the invention or of any of its preferred embodiments, may further comprise at least one of the following elements:
a. An external light source suitable for use in the Heatsens technology such as a laser or a LED light;
b. A second recognition molecule capable of recognizing the target analyte; or
c. A metal nanoparticle with photonic properties; and
d. Optionally a device capable of detecting the heat generated by the metal nanoparticles when they are irradiated with the external light source.

In an eighth aspect of the invention, the kit or device of the sixth aspect of the invention or of any of its preferred embodiments, may further comprise at least one of the following elements:
a. An external light source suitable for use in the Heatsens technology such as a laser or a LED light, preferably the external light source consists of a light-emitting diode (LED), wherein said light source is preferably emitting at between 600 nm and 1100 nm; or
b. A metal nanoparticle with photonic properties functionalized with a second recognition molecule capable of recognizing the target analyte; and
c. Optionally a device capable of detecting the heat generated by the metal nanoparticles when they are irradiated with the external light source.

In a ninth aspect of the invention, the kit or device of the sixth aspect of the invention or of any of its preferred embodiments, may further comprise at least one of the following elements:
a. An external light source suitable for use in the Heatsens technology such as a laser or a LED light;
b. A second recognition molecule (detection biomolecule) capable of recognizing the target analyte, optionally bound to a label molecule; or
c. Metal nanoparticles with photonic properties functionalized with biomolecules specifically recognizing the detection biomolecule or the label with which the detection biomolecule is modified; and
d. Optionally a device capable of detecting the heat generated by the metal nanoparticles when they are irradiated with the external light source.

Preferably the kit or a device of any of the seventh to eighth aspects of the invention, further comprises a device capable of detecting the heat generated by the metal nanoparticles when they are irradiated with the external light source selected from the list consisting of infrared cameras or thermopiles.

Sensor System Combining the Microchip Technology with the Heatsens Technology Suitable for Detecting the Presence of an Analyte in a Sample Fluid

Additional aspects of the present invention refer to a full sensor system which combines the microchip technology with the Heatsens technology.

Therefore, a tenth aspect of the invention refers to a device or system for detecting the presence of an analyte in a sample fluid, comprising:
a. a support or substrate;
b. a channel in the substrate, the channel comprising an inlet, an outlet, and a flowpath connecting the inlet and outlet, wherein the inlet and outlet together define a midplane; and a portion of the flowpath travels transversely across the midplane, wherein the portion of the flowpath that travels transversely across the midplane includes a sensing area comprising a recognition molecule (capture biomolecule) capable of recognizing the target analyte, thereon immobilized;
c. An external light source suitable for use in the Heatsens technology such as a laser or a LED light;
d. A second recognition molecule (detection biomolecule) capable of recognizing the target analyte;
e. A metal nanoparticle with photonic properties; and
f. Optionally a device capable of detecting the heat generated by the metal nanoparticles when they are irradiated with the external light source.

An eleventh aspect of the invention refers to a device or system for detecting the presence of an analyte in a sample fluid, comprising:
a. a substrate;
b. a channel in the substrate, the channel comprising an inlet, an outlet, and a flowpath connecting the inlet and outlet, wherein the inlet and outlet together define a midplane; and a portion of the flowpath travels transversely across the midplane, wherein the portion of the flowpath that travels transversely across the midplane includes a sensing area comprising a recognition molecule (capture biomolecule) capable of recognizing the target analyte, thereon immobilized;
g. An external light source suitable for use in the Heatsens technology such as a laser or a LED light;
c. A metal nanoparticle with photonic properties functionalized with a second recognition molecule (detection biomolecule) capable of recognizing the target analyte; and
d. Optionally a device capable of detecting the heat generated by the metal nanoparticles when they are irradiated with the external light source.

A twelfth aspect of the invention refers to a device or system for detecting the presence of an analyte in a sample fluid, comprising:
a. a substrate;
b. a channel in the substrate, the channel comprising an inlet, an outlet, and a flowpath connecting the inlet and outlet, wherein the inlet and outlet together define a midplane; and a portion of the flowpath travels transversely across the midplane, wherein the portion of the flowpath that travels transversely across the midplane includes a sensing area comprising a recognition molecule (capture biomolecule) capable of recognizing the target analyte, thereon immobilized;
c. An external light source suitable for use in the Heatsens technology such as a laser or a LED light;
d. A second recognition molecule (detection biomolecule) capable of recognizing the target analyte, optionally bound to a label molecule;
e. Metal nanoparticles with photonic properties functionalized with biomolecules specifically recognizing the detection biomolecule or the label with which the detection biomolecule was modified; and
f. Optionally a device capable of detecting the heat generated by the metal nanoparticles when they are irradiated with the external light source.

It is noted that the sensing area of the system or device of any of the tenth to twelfth aspects of the invention can be functionalized according to any of the techniques and with any of the functional groups described in the section entitled “USE OF THE MICROFLUIDIC TECHNOLOGY IN COMBINATION WITH THE HEATSENSE TECHNOLOGY”.

Preferably, the functionalization used allows an oriented configuration of the recognition molecule, preferably of an antibody.

In addition, it is further noted that the microchip or device mentioned as one of the components of the full sensor system of any of the tenth to twelfth aspects of the invention, may be further characterized as described in any of the embodiments described in the section entitled “USE OF THE MICROFLUIDIC TECHNOLOGY IN COMBINATION WITH THE HEATSENSE TECHNOLOGY”.

Processes for Functionalizing the Sensing Area of a Microchip or Device Suitable for Carrying Out Immunoassays by Detecting an Analyte by Using the Heatsens Technology.

As illustrated in the examples (see examples 6 to 9), functioanalizing the sensing area of the microchip's surface improves the characteristics of the sensor by providing a covalent or oriented configuration of the capture biomolecule.

As already established in the section entitled “USE OF THE MICROFLUIDIC TECHNOLOGY IN COMBINATION WITH THE HEATSENSE TECHNOLOGY” or in the section entitled “MICROFLUIDIC DEVICE OR CHIP HAVING A SENSING AREA FUNCTIONALIZED FOR AN ANTIBODY ORIENTED IMMOBILIZATION”, the sensing area of a microchip or device for use in carrying out immunoassays by detecting an analyte by using the Heatsens technology, can be functionalized in a number of different ways. The different ways of functionalizing the microchip or device depend on the type of material to functionalize and on the type of organic functional groups (such as carboxylic functional groups or epoxy functional groups or amine functional groups or thiol functional groups or azide functional groups or halides or maleimide functional groups or hydrazyde functional groups or aldehydes or alkynes groups) with which we wish to functionalize the sensing area of any of the microchips or devices shown through-out the present invention.

In this sense, it is noted that the substrate or surface of the microchip or device may be made from a variety of materials such as thermoplastic materials, silicon, metals, or carbon. Preferably, it may be made by poly(methyl methacrylate), polystyrene, poly(dimethylsiloxane), polyethylene terephthalate, polyethylene, polypropylene, polylactic acid, poly(D,L-lactide-co-glycolide), polycarbonate, cyclic olefin copolymers, silicon, glass etc.

Different manners of functionalizing these types of surfaces with the above mentioned functional groups are shown in the examples. In this sense, a thirteenth aspect of the invention refers to a process for functionalizing the sensing area of a microchip or device comprising a support or substrate, wherein said support or substrate comprises at least one channel in the substrate, the channel comprising an inlet, an outlet, and a flow-path connecting the inlet and outlet, wherein the inlet and outlet together define a midplane; and a portion of the flowpath travels transversely across the midplane, wherein the portion of the flowpath that travels transversely across the midplane includes a recognition site or sensing area for detecting a target analyte;

wherein if the support or substrate is made of:
a. a thermoplastic material such as a co-olephin polymer, functionalization is carry out by using a diazonium aryl compound containing one or more carboxylic groups or epoxy groups or amine groups or thiol groups or azide groups or halides or maleinido functional groups or hydrazyde functional groups or aldehydes or alkynes groups;
b. silicon material such as polydimethylsiloxane (PDMS) or glass, functionalization is carry out through self-assembly with organo-functional alkoxysilane molecules carrying one or more carboxylic groups or epoxy groups or amine groups or thiol groups or azide groups or halides or maleinido functional groups or hydrazyde functional groups or aldehydes or alkynes groups;
c. a metal such as iron, cobalt, nickel, platinum, palladium, zinc, silver, copper or gold, functionalization is carry out through self-assembly with molecules capable of interacting with the metal, such as thiol groups in the case of gold and silver, carrying one or more carboxylic groups or epoxy groups or amine groups or thiol groups or azide groups or halides or maleinido functional groups or hydrazyde functional groups or aldehydes or alkynes groups;
d. a carbon material such as graphene, functionalization is carry out as established in step a) above or through an oxidation to generate aldehydes and carboxylic functional groups or through hydrophobic binding of functionalized polymers having one or more carboxylic groups or epoxy groups or amine groups or thiol groups or azide groups or halides or maleinido functional groups or hydrazyde functional groups or aldehydes or alkynes groups.

Preferably, if the support of the microfluidic chip or device is made of a thermoplastic material the diazonium aryl compound is represented by formula I or II below:

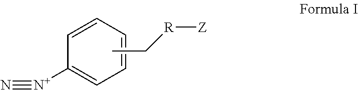

wherein R is an alkyl group having from 1 to 15 carbon atoms or an ethylene; and
Z is a carboxylic group, an epoxy group, an amine group, a thiol group, an azide group, a halide, a maleinido functional group, a hydrazyde functional group, an aldehyde group or an alkyne group, preferably a carboxylic or epoxy group;

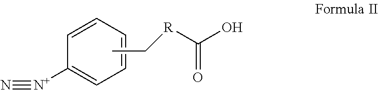

wherein R is an alkyl group having from 1 to 15 carbon atoms.

Preferably the diazonium component of formula I or II above is place or sited in the para or meta position with respect to the alkyl or ethylene component of any of these formulae. Examples of aryl amine compounds suitable for producing the diazonium aryl compound of any of formula I or II above are: 3-(4-Aminophenyl)propionic acid, 3-Aminophenylacetic acid, 4-Aminophenylacetic acid, 4-(4-Nitrophenyl)butyric acid or 4-(4-Aminophenyl)butyric acid.

In a further preferred embodiment of the thirteenth aspect of the invention, the surface of the microchip or device is further modified or functionalized with a chelating agent preferably selected from the list consisting of: Nα,Nα-Bis(carboxymethyl)-L-lysine hydrate (ANTA) metal (II) salt, nitrilotriacetic acid (NTA) metal (II) salt, iminodiacetic acid (IDA) metal (II) salt, Ethylenediaminetetraacetic acid (EDTA) metal (II) salt, diethylenetriaminepentaacetic acid (DTPA) metal (II) salt, wherein metal (II) salt is understood as a salt of a divalent metal such as Cu2+, Ni2+ or Co2+. This is accomplished by direct reaction of the chelating agent with any of the activated functional groups referred to above, wherein:
carboxylic groups can be activated via EDC/SNHS-mediated amidation (Scheme III);
amine groups can be activated with carbonyl groups;
thiol groups can be activated by forming sulfhydryl-reactive crosslinkers, wherein sulfhydryls can be selected from maleimides, haloacetyls or pyridyl disulfides;
alkyne or azide groups can be activated through CLICK chemistry;
aldehyde groups can be activated via the shift base formation.

Preferably, the support is made of a thermoplastic material and the aryl amine compounds contain carboxylic groups activated via esterification with N-Hydroxysulfosuccinimide salt (Sulfo-NHS) catalyze by (N-(3-Dimethylaminopropyl)-N′-ethylcarbodiimide hydrochloride (EDC).

More preferably, the support is made of a thermoplastic material, the aryl amine compounds contain carboxylic groups activated via esterification with N-Hydroxysulfosuccinimide salt (Sulfo-NHS) catalyze by (N-(3-Dimethylaminopropyl)-N′-ethylcarbodiimide hydrochloride (EDC), and the chelating agent is selected from the list consisting of: Nα,Nα-Bis(carboxymethyl)-L-lysine hydrate (ANTA) metal (II) salt or nitrilotriacetic acid (NTA) metal (II) salt, wherein metal (II) salt is understood as a salt of a divalent metal such as Cu2+, Ni2+ or Co2+, preferably Cu2+.

As illustrated in the examples, activation of the microchip's surface with a chelating agent such as ANTA metal (II) salt is especially advantageous to achieve an oriented configuration of the antibody resulting in an improved sensing platform.

Kit or Device Having a Sensing Area Functionalized for an Antibody Oriented Immobilization.

Lastly, it is noted that, as illustrated in the examples, by functionalizing the sensing area of any support, not necessarily the support of a microchip or device, such as glass, so that a capture biomolecule, such as an antibody, has an oriented configuration provides clear advantages for the detection of an analyte in a sensor system which uses the Heatsens technology.

Thus, a fourteenth aspect of the invention refers to a kit or device comprising a support or substrate, wherein said substrate or surface may be made from a variety of materials such as thermoplastic materials, silicon, metals, or carbon; wherein said support or substrate includes a recognition site or sensing area for detecting a target analyte; and wherein said recognition site or sensing area is functionalized with a chelating agent.

In a preferred embodiment of the fourteenth aspect of the invention, the chelating agent is preferably selected from the list consisting of: Nα,Nα-Bis(carboxymethyl)-L-lysine hydrate (ANTA) metal (II) salt, nitrilotriacetic acid (NTA) metal (II) salt, iminodiacetic acid (IDA) metal (II) salt, Ethylenediaminetetraacetic acid (EDTA) metal (II) salt, diethylenetriaminepentaacetic acid (DTPA) metal (II) salt, wherein metal (II) salt is understood as a salt of a divalent metal such as Cu2+, Ni2+ or Co2+. The chelating agent functionalizes the support by direct reaction of the chelating agent with an activated functional group, wherein:
carboxylic groups can be activated via EDC/SNHS-mediated amidation (Scheme III);
amine groups can be activated with carbonyl groups;
thiol groups can be activated by forming sulfhydryl-reactive crosslinkers, wherein sulfhydryls can be selected from maleimides, haloacetyls or pyridyl disulfides;
alkyne or azide groups can be activated through CLICK chemistry;
aldehyde groups can be activated via the shift base formation.

It is noted that in the section entitled “PROCESSES FOR FUNCTIONALIZING THE SENSING AREA OF A MICROCHIP OR DEVICE SUITABLE FOR CARRYING OUT IMMUNOASSAYS BY DETECTING AN ANALYTE BY USING THE HEATSENS TECHNOLOGY”, we described how to functionalize different supports or surfaces with any of the organic functional groups mentioned through-out the present invention.

Preferably, the support is made of glass functionalized via self-assembly with organo-functional alkoxysilane molecules carrying one or more carboxylic groups or epoxy groups or amine groups or thiol groups or azide groups or halides or maleinido functional groups or hydrazyde functional groups or aldehydes or alkynes groups; wherein said functional groups have been optionally activated and directly reacted with a chelating agent, preferably with a chelating agent selected from the list consisting of: Nα,Nα-Bis(carboxymethyl)-L-lysine hydrate (ANTA) metal (II) salt or nitrilotriacetic acid (NTA) metal (II) salt, wherein metal (II) salt is understood as a salt of a divalent metal such as Cu2+, Ni2+ or Co2+, preferably Cu2+.

In a further preferred embodiment of the fourteenth aspect of the invention or of any of its preferred embodiments, the recognition site or sensing area may comprise:
a. a recognition molecule capable of recognizing a target analyte immobilized onto the recognition site or sensing area; or
b. a target analyte immobilized onto the recognition site or sensing area.

Preferably, said recognition molecule can be selected from, but not limited to, the list consisting of: peptides, polysaccharides, toxins, protein receptors, lectins, enzymes, antibodies, antibody fragments, recombinant antibodies, antibody dendrimer complexes, nucleic acids, (DNA, RNAs), peptide nucleic acids (PNAs), molecular imprints. Preferably, said recognition molecule is an antibody, a fragment thereof or a recombinant antibody.

In a fifteenth aspect of the invention, the kit or device of the fourteenth aspect of the invention or of any of its preferred embodiments, may further comprise at least one of the following elements:
a. An external light source suitable for use in the Heatsens technology such as a laser or a LED light;
b. A second recognition molecule capable of recognizing the target analyte; or
c. A metal nanoparticle with photonic properties; and
d. Optionally a device capable of detecting the heat generated by the metal nanoparticles when they are irradiated with the external light source.

In a sixteenth aspect of the invention, the kit or device of the fourteenth aspect of the invention or of any of its preferred embodiments, may further comprise at least one of the following elements:
a. An external light source suitable for use in the Heatsens technology such as a laser or a LED light; or
b. A metal nanoparticle having photonic properties; and
c. Optionally a device capable of detecting the heat generated by the metal nanoparticles when they are irradiated with the external light source.

In a seventeenth aspect of the invention, the kit or device of the sixth aspect of the invention or of any of its preferred embodiments, may further comprise at least one of the following elements:
a. An external light source suitable for use in the Heatsens technology such as a laser or a LED light;
b. A second recognition molecule (detection biomolecule) capable of recognizing the target analyte, optionally bound to a label molecule; or
c. Metal nanoparticles with photonic properties functionalized with biomolecules specifically recognizing the detection biomolecule or the label with which the detection biomolecule was modified; and
d. Optionally a device capable of detecting the heat generated by the metal nanoparticles when they are irradiated with the external light source.

Preferably the kit or a device of any of the fifteenth to seventeenth aspects of the invention, further comprises a device capable of detecting the heat generated by the metal nanoparticles when they are irradiated with the external light source selected from the list consisting of infrared cameras or thermopiles.

Lastly, an eighteenth aspect of the invention refers to the in vitro use of the kit or device of any of the fourteenth to seventeenth aspects of the invention for detecting an analyte as a result of the heat generated by metal nanoparticles when they are irradiated with an external light source.

The invention has been described broadly and generically herein. Each of the narrower species and sub-generic groupings falling within the generic disclosure also form part of the invention. This includes the generic description of the invention with a proviso or negative limitation removing any subject matter from the genus, regardless of whether or not the excised material is specifically recited herein.

Other embodiments are within the following claims and non-limiting examples. In addition, where features or aspects of the invention are described in terms of groups, those skilled in the art will recognize that the invention is also thereby described in terms of any individual member or subgroup of members of the group.

### EXAMPLES

### Materials and Methods

Fabrication of the Microchip.

Sandwich immunoassays for the detection of pathogens such as Salmonella and Campylobacter, allergens such as the Ara h1, and other protein molecules such as albumin and collagen, have been implemented in microfluidic chips in the present invention. They were properly sketched and fabricated, as displayed in FIG. 1, to fulfil the following target specifications:
1. The use of thermoplastic material (co-olephin polymer) for the fabrication of the microfluidic chip, in particular, the use of a film with a thickness in a range between 50 and 150 μm. The microchip used in the present examples was construed by using a thermoplastic material having a thickness of about 100 μm on the side where the temperature was monitored.
2. The integration of micro-chambers with approximate dimensions of 5×3 mm and 0.1 mm in depth and a total volume between 1 and 3 μl. The volume of the microfluidic chamber being of 1 μl.
3. Each micro-chamber should be at least 10 mm further apart from each other.
4. An easy-to-use design to allow the insertion of at least 3 different liquids.
5. The integration of 5 different detection channels
   1× Control, which is also the calibration curve:
      Including 5 microchambers in-line per detection channel. All microchambers placed in line, including dedicated inlets and outlets for each microchamber.
   4× Detection:
      Including 3 micro-chambers in-line per detection channel.
      Each detection channel with dedicated outlets.

The final chip design includes three dedicated inlets to allow a straight forward insertion of the reagents (see FIG. 1). The detection assay is then split in 5 different micro-channels. The one placed at the top of the cartridge is dedicated for performing a calibration protocol. It includes 5 different micro-chambers with known concentrations of the analyte. The other 4 micro-channels are used for the assay itself, allowing the use of different samples and internal controls. In order to avoid contamination problems, each sample was injected using a dedicated inlet. In addition, each sample micro-channel was designed to include 3 equal micro-chambers to perform statistical relevant assay replicates.

The layout of the microfluidic chip, was originally conceptually designed for the specific detection of pathogens present in the poultry sector, even though the microchip referred to herein was also successfully applied for the detection of other biomolecules such as the Ara h1, collagen and albumin. In this sense, the present invention is not limited to the specific layout of the microfluidic chip described herein.

Reagents for the Different Functionalizations of the Microchip

4-(4-Aminophenyl)butyric acid (PhBut), sodium nitrite (NaNO2), hypophosphorous acid (H3PO2, 50 wt. % in H2O), (N-(3-Dimethylaminopropyl)-N′-ethylcarbodiimide hydrochloride (EDC), N-Hydroxysulfosuccinimide sodium salt (Sulfo-NHS), Nα,Nα-Bis(carboxymethyl)-L-lysine hydrate (ANTA), copper (II) sulfate, 20 mM HEPES buffer pH 8.0, 2.5 N NaOH solution, 1 N HCl solution, absolute ethanol (EtOH), distilled Type-I water (>18.2 Mohm-1). Streptavidin Ref 7100-05 Lote A2805-PA05H. 2% solution of 3-(2,3-epoxypropoxy)propyltrimethoxysilane in dry toluene. 10 mM carbonate buffer pH 10.8. 10 mM MES (2-(N-morpholino)ethanesulfonic acid) buffer, pH 5. Piranha solution (H₂SO₄:H₂O₂ 3:1)

Reagents for the Detection of Salmonella thiphymorium

a. Capture antibody: Anti-Salmonella typhimurium 0-4 antibody [1E6] ab8274-Abcam 2 mg/mL.
b. 5 μg/mL disuelto en PBS 1×.
c. Blocking: TBS-T 0.1%+BSA 5%.
d. Antigen: BacTrace Salmonella typhimirium positive control Ref 50-74-01-KPL. Cell count: 3×10⁹ CFU/mL.
e. Detection antibody: Anti-Salmonella antibody (Biotin) ab69255-Abcam 4 mg/mL
f. Dilution 1/5.000 dissolved in TBS-T 0.1%+BSA 5%.

Reagents for the Detection of Campylobacter jejuni

a. Capture antibody: Anti-Campylobacter jejuni antibody ab155855 Lote: GR146930-4 0.1 mg/mL.
b. Dilution 1/20=5.0 μg/mL PBS1×.
c. Antigen: BacTrace Campylobacter jejuni positive control Ref 50-92-93. Lot 140513-KPL Cell count: 4.64×10⁸ CFU/mL.
d. Detection antibody: Anti-Campylobacter jejuni antibody-Biotin ab53909 Lot GR93260-3.
e. Dilution 1/1000 TBS-T 0.1%+BSA 5%.

Reagents for the Detection of Ara h1

a. Capture antibody: Monoclonal antibody 2C12 Mouse IgG1 Lot: 30083 2.7 mg/mL
b. Dilution 1/500=5.4 μg/mL PBS1×.
c. Allergen Standard nArah1 Ref EL-AH1-Standard. Lot 38018 20.000 ng/mL.
d. Detection antibody: Monoclonal Antibody 2F7 Mouse IgG-Biotinylated Lot 36069.
e. Dilution 1/1000 PBS-T 0.1%+BSA 5%.
f. Dilution 1/2000 PBS-T 0.1%+BSA 5%.

Reagents for the Detection of Albumin and Collagen

a. The OVA polyclonal antibody: Goat Anti-Rabbit IgG H&L (Biotin), Abcam ref: ab6720
b. Monoclonal Anti-chicken egg albumin (ovalbumin) antibody produced in mouse, Sigma ref: A6075
c. Mouse monoclonal to collagen I, GeneTex ref: GTX26308
d. Rabbit polyclonal to collagen I (Biotin), Genetex ref: GTX26577

### Example 1. Microfluidic Chip Surface Functionalization with Carboxylic Groups by Covalent Grafting of Diazotated PhBut

Surface functionalization with carboxylic groups is obtained by covalent grafting of the aryl radical of diazotated PhBut (Scheme I) generated by both chemical reduction (H₃PO₂) and UV radiation that bonds to the chamber chip's surface (Scheme II).

### Procedure

1. Diazotation of PhBut

Diazotated PhBut is obtained in situ previous to its use in an ice bath by dissolving the amount of NaNO₂ to reach a 0.3 M final concentration, in a 0.1 M PhBut solution prepared in 0.5 M HCl. This mixture is held at 4° C. for 10 min before use in surface modification.

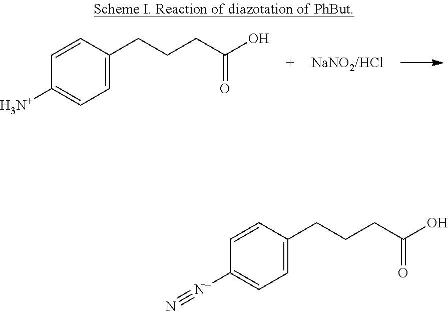 [SMILES: [NH3+]c1ccc(CCCC(=O)O)cc1 ; N#[N+]c1ccc(CCCC(=O)O)cc1]

2. Covalent Grafting of Diazotated PhBut.

Prior the surface modification, the chips are rinsed with ethanol and dried. Then, they are irradiated for 15 minutes with ultraviolet (UV in the range between 305 and 395 nm) light by exposing under UV-lamp (8 W) at wavelength of 365 nm. Diazotated PhBut solution just prepared as above described is mixed with H₃PO₂ acid solution to reach a 0.16 M final concentration and drop casted on the chip's chambers. They are again placed under UV-lamp and irradiated for 30 min at a wavelength of 365 nm. Finally, the modified chips are removed from the lamp and extensively rinsed with absolute EtOH.

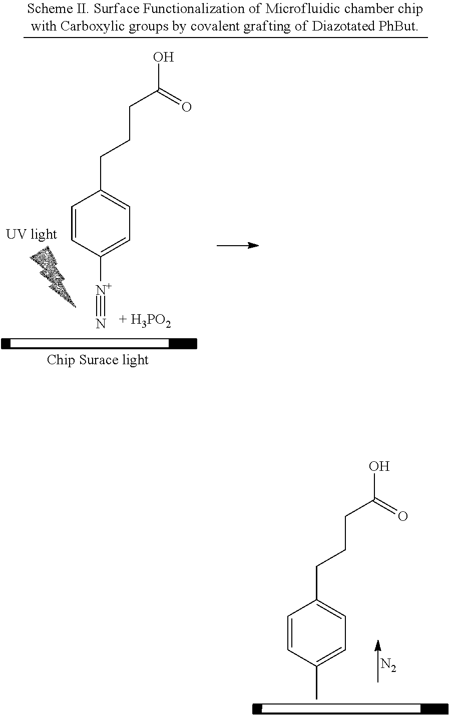

### Example 2. Microfluidic Chamber Chip Modification of Carboxylic-Acid Terminated Surface with Nitrilotriacetic-Cu(II)

NTA-Cu(II) surface modification is accomplished by activating carboxylic groups and direct reaction with primary amine (—NH2) of ANTA via EDC/SNHS-mediated amidation (Scheme III).

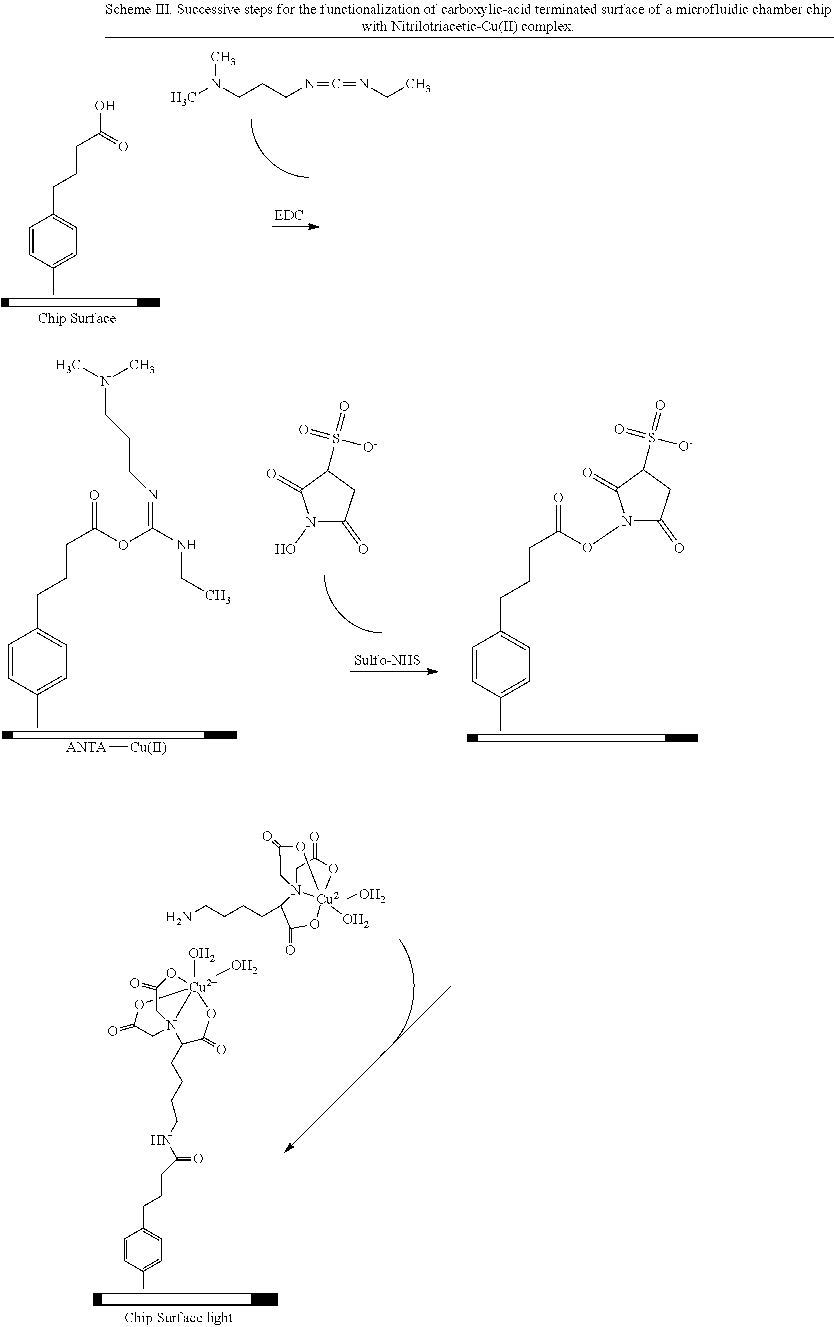

Activation of the carboxylate groups of the surface-modified chambers and subsequent amidation of the NHS-esters with the ANTA-Cu(II) complex is performed in several steps. Firstly, a 20 mM SNHS and 10 mM EDC solution is prepared by dissolving sulfo-NHS reagent in distilled Type-I water and transfer to EDC reagent. This solution containing the reagents is drop casted on the chip's chambers and allowed to react for 1 h at room temperature. Following, the chips are rinsed with distilled Type-I water and incubated in a solution of 25 mM ANTA in 10 mM sodium bicarbonate solution, pH 10 overnight to introduce the chelate. Finally, after removing the reagent excess by washing with water and dried, nitrile-tri-acetic-Cu(II) complex (ANTA-Cu2+) is formed on the surface by incubation of the chip's chambers in a 100 mM copper (II) sulfate aqueous solution for 3 hours. The chips are again wash and dried being ready for antibody immobilization.

### Example 3. Microfluidic Chamber Chip Modification of Carboxylic-Acid Terminated Surface with Other Nitrilotriacetic-M(II) (Ni2+, Co2+) Complexes

The surface modification with others NTA-M2+ complexes can be also accomplished following the same procedure as for NTA-Cu(II) employing instead of CuSO₄, the corresponding metal salt (CoCl₂, NiSO₄ or NiCl₂) in similar concentrations as above described. The binding affinity of the NTA-chelated metal atom towards histidine-tagged proteins and antibodies follows the order Cu(II)>Ni(II)>Co(II).

### Example 4. Functionalization of Glass Surfaces

Two types of bio-functionalization of glass surfaces have been performed, covalent non-oriented and oriented immobilization. To activate glass supports, surfaces are cleaned with piranha solution for 1 hour at room temperature in an orbital shaker. Subsequently, slides are rinsed with milli-Q water and dried. Then, 2% solution of 3-(2,3-epoxypropoxy)propyltrimethoxysilane in dry toluene is added overnight at room temperature in an orbital shaker onto the activated glass supports. Afterwards, slides are washed thoroughly with toluene and 10 mM carbonate buffer pH 10.8. After drying the slides, glass supports are incubated with 25 mM NTA for 3 hours at room temperature in an orbital shaker. Later, glass supports are washed extensively with 10 mM carbonate buffer at pH 10.8.

In order to have an oriented immobilization, NTA-surfaces are incubated overnight with 100 mM CuSO₄ in aqueous solution at room temperature for complexation environment. Then, slides are washed with milli-Q water. For a non-oriented covalent immobilization, NTA-surfaces are loaded with 50 mM EDC and 75 mM SNHS in 10 mM MES pH 5 for 45 min at RT for further carboxyl group activation. Then, surfaces are washed with 10 mM MES pH 5.

### Example 5. Surface Functionalization—Microfluidic Chamber Chip Modification with Capture Antibodies

1. Physical Absorption

Prior to the antibody surface modification, the chips are rinsed with EtOH and dried. Then 5 μl of 5 μg/ml of capture antibodies in PBS 1× are casted only onto the surface of the Microfluidic chamber (sensing area) inside the microfluidic channel and incubated at 37° C. for one hour.

The surface is rinsed with PBS 1× and incubated over night at 4° C. with blocking buffer (BSA 5% in PBS1×, 0.1% tween).

The surface is washed and the chip is assembled with the upper part (PMMA) and connected to the peristaltic pump.

2. Carboxylated-Functionalized Microfluidic Chamber Chip Surface Modification with Capture Antibodies: Covalent Antibody Immobilization

The activation of the carboxylate groups of the surface-modified chambers and subsequent amidation of the NHS-esters with the capture antibodies is performed in a two-step process as described as follows:
1. Incubation of carboxylated-microchamber with 10 μl of 20 mM SNHS and 10 mM EDC, in 10 mM MES buffer (pH 6) for 10 minutes.
2. Wash with 10 mM MES at a pH 6 and incubation with 10 μl of 5 μg/ml of capture antibodies for 1 hour at 37 C, on each micro-chamber.

After the covalent immobilization of the capture antibodies and the blocking of surface with BSA 5% in PBS1×/0.1% Tween for 1 hour at 37° C., the chip is connected to the peristaltic pump and each channel is rinsed with washing buffer using a flow rate of 300 μl/min for 4 minutes.

3. Modification of Nitrilotriacetic-M(II) (Cu2+, Ni2+, Co2+) Complexes Functionalized Microfluidic Chamber Chip with Capture Antibodies: Oriented Antibody Immobilization

Modification with capture antibodies onto a NTA-M(II) (Cu2+, Ni2+, Co2+)-functionalized Microfluidic chamber chip is carried out in a single step, as described as follows: 5 μl of 5 μg/ml of capture antibodies are deposited only on the surface of the sensing area of the microfluidic channel and incubated for at 37° C. for one hour.

Then the surface is rinsed and incubated over night at 4° C. with blocking buffer (BSA5% in PBS 1×, 0.1% tween). Following, the surface is rinsed and the chip is assembled with the upper part (PMMA) and connected to the peristaltic pump.

### Example 6. Immunoassays Using a Microfluidic Chip

In the following we illustrate different immunoassays implemented in the microchip referred to in the materials and methods for the detection of Salmonella. In addition, we have also compared the results obtained with these methods.

1. Direct Immunoassay for Salmonella Detection: Temperature Increment of First Test Using Chips Directly Functionalized with Two Different Dilutions of Salmonella

The unmodified fabricated microfluidic chip illustrated in the materials and method was used for testing the direct immobilization of two dilutions of salmonella.

10 μl of 60000 CFU/ml and 20000 CFU/ml (600 and 200 CFU in total on the surface, respectively) of Salmonella T. were adsorbed on the detection surface. After the direct immobilization of the pathogen, the surface was blocked with BSA and left to react with biotinylated detection antibodies. Finally, they were washed and further reacted with streptavidin-AuNanoprisms solution.

In order to test the specificity of the immunoassay, the following control experiments were performed: 1) NC1=absence of Salmonella, surface blocked with BSA 5%; 2) NC2=absence of biotinylated detection antibody; 3) NC3=absence of streptavidin-AuNPrisms.

In FIG. 2, it is reported the increment of temperature measured upon NIR irradiation of the surface due to the presence of the Salmonella after its recognition by biotinylated detection antibodies and further interaction with streptavidine-Nanoprisms.

In absence of biotinylated detection antibody (NC2) there is an insignificant increment in temperature as in absence of strepavidin@AuNPrism (NC3).

The increment of temperature is proportional to the amount of salmonella's CFUs. These results indicate the suitability of this material for the fabrication of the microfluidic chip and its application for HEATSENS. Moreover, the results envisage the possibility of immobilizing salmonella at different CFU dilutions directly onto a microfluidic chip and build a calibration curve.

2. Direct Immunoassay for Salmonella Detection: □Temperature Increment of First Test of Direct Immobilization of Salmonella and Detection of Two Different Dilutions of Salmonella on a Microfluidic Chip. Calibration Curve Test Construction.

10 μl of different concentrations (CFU/ml) of salmonella T, in a range between 0 and 240000 CFU/ml, were directly adsorbed onto the microfluidic chip and detected with biotinylated antibodies anti-salmonella to measure the increment of temperature due to the presence of different concentrations of salmonella. Then the strepavidine@AuNprism interacted with the antibodies and every single sensing area was irradiated with an IR laser. The temperature of each chamber was measured, and the increment of temperature calculated. FIG. 3 displays the calculated increment of temperature in function of the amount of salmonella's CFU/ml.

The increase of temperature measured was due to the increased amount of CFUs directly adsorbed onto the surface of microfluidic chip.

3. Sandwich Immunoassay for Salmonella Detection: Q Temperature Increment of First Test of Sandwich Immunoassay Detection of Two Different Dilutions of Salmonella on a Microfluidic Chip

Once shown that the microfluidic chip is suitable to be applied to the HEATSENS technology, we performed a sandwich immunoassay for the detection of the selected pathogen by using a microfluidic chip. For this purpose, each micro-chamber of the microchip was functionalized with capture antibodies anti-salmonella by direct adsorption of (5 μL) 5 μg/ml of capture antibodies anti-salmonella onto the surface. Then, the salmonella's capture event was carried out in fluidic mode, as well as the detection and the interaction with the streptavidin-AuNprism, injecting 1 ml of sample, in each channel.

The assay was carried out with 2 different concentrations of salmonella's CFU/ml, 200000 CFU/ml and 240000 CFU/ml diluted in buffer phosphate, respectively. FIG. 4 describes the trend of the increments of temperature due to the presence of Salmonella T.

The trend of the calibration curve is not linear, indicating a saturation of the signal due to the presence of high amount of nanoprisms interacting with the analyte. The detection of the two unknown concentrations of salmonella was calculated from the exponential equation, where the values concur with the curve with an adj. R-Square equal to 0.98843.

Once shown the effectiveness of an immunoassay in a sandwich format, we tried to improve the limit of detection of salmonella t., by decreasing the concentration of the pathogen in doped buffer.

1500 CFU/ml of salmonella T. in PBS 1× was the first lower concentration detected in the first trial.

1 ml of sample was injected in the channel with a flow of 200 μl/ml. After injecting the sample, the channel was washed with washing buffer (BSA 0.5% in PBS1×, 0.1% tween), using a flow of 300 μl/min for 4 min. Then the detection antibodies were left to interact with its antigen using a 200 μl/ml for 2 minutes. The channel was rinsed with washing buffer (BSA 0.5% in PBS1×, 0.1% tween), using a flow of 300 μl/min for 4 min. The streptavidin@AuNPr were injected into the channel. The flow was 200 μl/ml for 2 minutes. The channel was rinsed with washing buffer (BSA 0.5% in PBS1×, 0.1% tween), using a flow of 300 μl/mim for 4 min and dried.

FIG. 5 illustrates the increment of temperature of 1500 CFU/ml of salmonella with respect to the negative controls. The increment of temperature of the micro-chambers in the presence of salmonella was higher that the temperature increments of the controls, respectively in absence of salmonella (NC1), absence of detection antibodies (NC2), and absence of strepavidine-AuNPrism (NC3).

The temperature increment due to the presence of salmonella was higher than all negative controls, even though different from the expected value: the positive values of increment of temperature of the negative controls indicated non-specific interactions between the reagents within the immunoassay. The non-specific interactions can be associated to an uncompleted functionalization and blocking of the surface or to an inappropriate flow rate during the immunoassay. In this way, by keeping constant the surface antibody functionalization and modifying the flow rate during the immunoassay, it was possible to improve the limit of detection of salmonella and the signal due to the background, as shown in FIG. 6.

The same experiment was carried out using a real food sample, 25 μg of chicken meat in 225 ml of peptone pre-enrichment culture media, doped with salmonella at different CFUs. The capture antibodies were adsorbed onto the microfluidic chip, and the surface blocked with 5% BSA in PBS1×-01% Tween, using a flow rate of 150 μl/min.

Then, the washing was carried out using a flow rate of 250 μl/min, by using a washing buffer.

The capture of salmonella in 1 ml of real sample, as well as the detection with biotinylated detection antibodies, and the interaction with streptavidin@nanoprisms was performed by using a flowing at a flow rate of 15 μl/min.

The results of the immune analysis carried out in the microfluidic chip are shown in FIG. 7.

After building the calibration curve, measuring the increment of temperature due to the known different concentrations of salmonella, the unknown concentration of pathogen in the real sample was determined from the calibration curve (FIG. 8).

The higher increment of temperature of the samples doped with salmonella, clearly indicates that HEATSENS is suitable for the ultrasensitive detection of few CFUs of bacteria in complex matrices such as the 25 g of chicken meat in 225 ml of peptone.

The increment of temperature due to the presence of salmonella in a real sample is slightly different from the one in buffer phosphate, because of presence of high amount of meat proteins which affect the specific interaction of the bacteria with the antibodies.

4. Sandwich Immunoassay for Salmonella Detection: Effect of Covalent Immobilization of the Capture Ab on Microfluidic Chip

The modification of a microfluidic chip surface with carboxylic end group can be used to immobilize covalently capture antibodies by formation of stable amide bonds with their primary amines via EDC/sulfo-NHS reaction.

In this sense, the surface of each micro-chamber, previously activated with 10 mM EDC and 20 mM sulfo-NHS, was functionalized with 20 μl of 5 μg/ml of capture antibodies. After the covalent immobilization of the capture antibodies and the blocking of surface with BSA 5% in PBS1×/0.1% Tween for 1 hour at 37° C., the chip was connected to the peristaltic pump and washed with washing buffer using a flow rate of 300 μl/min for 4 minutes. 1 ml of 30 CFU/ml of Salmonella T, were allow to flow inside the microfluidic channel for 1 minute at a flow rate of 150 μl/min, then the channel was washed with a buffer solution using a flow rate of 300 μl/min for 4 minutes. 400 μl of biotinylated detection antibodies were then flowed inside the channel.

The results depicted in FIG. 9 show that the temperature increment in the sample doped with 30 CFU/ml of Salmonella was higher in comparison with those of different controls. In this type of immobilization, the antibody adopts a predominantly “flat-on” orientation with the Fc and two Fab fragments lying flat on the surface.

5. Sandwich Immunoassay for Salmonella Detection: Oriented Immobilization of Capture Antibodies Through Metal-Chelation on Microfluidic Chip.

Oriented immobilization of antibodies through metal-chelation constitutes an optimal and versatile method as shown herein. Immobilization is accomplished through the metal-chelation to histidine-rich metal binding site in the heavy chain (Fc) of the antibody or to poly-His-tag sequence fused in proteins. Since the metal binding site is either in the C- or N-terminus, antibodies and His-tagged proteins bound in this fashion to the surface are oriented with the combining site directed away from the surface thus allowing maximal antigen binding or a favourable protein orientation. Furthermore, oriented immobilization through metal-chelation also results in a stable antibody immobilization since binding constants for metal-chelation immobilization are very high due to the combination of the chelate effect of histidine binding, and target binding of multiple metal-chelate groups. Dissociation constants are estimated to be between 10⁻⁷ to 10⁻¹³ M⁻¹. For many applications, this provides binding strengths comparable to antigen-antibody interaction. On the other side, experimental conditions of antibody attachment for oriented immobilization of antibodies through metal-chelation are milder than those employed for covalent oriented immobilization procedure. As an advantage, the antibody binding to the chelate could be also modulated as convenience to be reversible or irreversible. In addition, it is also more versatile since it can be also employed for immobilization of his-tagged recombinant proteins.

In order to achieved an oriented immobilization of the capture antibodies, the microfluidic chamber chips were functionalized with metal-chelate complexes in a stepwise modification of their surface. Firstly, the surfaces were functionalized with aryl amine compounds containing carboxylic groups such as for example 3-(4-Aminophenyl)propionic acid, 3-Aminophenylacetic acid, 4-Aminophenylacetic acid or 4-(4-Nitrophenyl)butyric acid. For this specific example we used PhBut, even though for the immobilization of different biomolecules, it would be more appropriate the use of aryl amine compounds carrying different lengths of n-alkyl carboxylic acids in a range between 2 and 16 carbons.

Carboxylic groups introduced by covalent grafting of the aryl radical of diazotated PhBut (Scheme II) were activated by esterification with SNHS catalyzed by EDC to facilitate the covalent linkage of the ANTA-M(II) (Cu2+, Ni2+, Co2+) complex (Scheme III) through the free amino groups. Then, they were incubated with 20 μl of 5 μg/ml of capture antibodies. The resulting NTA-M(II) complex termination contains two free coordination sites occupied by water molecules to be replaced by histidine residues of capture antibodies giving rise to their oriented immobilization. Later, the chip was connected to the peristaltic pump and washed with washing buffer using a flow rate of 300 μl/min for 4 minutes. 1 ml of 30 CFU/ml of salmonella T., was allow to flow inside the microfluidic channel for 1 minute at a flow rate of 150 μl/min, then the channel was washed with buffer using a flow rate of 300 μl/min for 4 minutes. 400 μl of biotinylated detection antibodies was then flowed inside the channel.

FIG. 10 illustrates the detection of salmonella on a microfluidic chip functionalized with capture antibodies in an oriented manner.

Interestingly, the temperature increment due to the presence of Salmonella for this type of immobilization was higher than those obtained for the respective controls and even higher than those obtained in previous results for direct adsorption and covalent immobilization. In this sense, a comparative study between the different immobilization methods was carried out. The comparison of the different strategies of antibody surface functionalization is displayed in the FIG. 11, where it is shown the increment of temperature due to the detected Salmonella in comparison with the generated background signal, for each of the surface functionalization strategies shown in this example.

FIG. 11 shows that the oriented immobilization of capture antibodies through metal-chelation provides the best results by providing the highest temperature increment due to the presence of salmonella and by providing the lowest signal generated by non-specific interactions (background). These results indicate that a correct functionalization strategy of the surface of the chip is crucial in order to obtain an optimal antibody attachment in a favorable orientation, while avoiding non-specific adsorptions of HEATSENS labels (gold nanoprisms). It is also noteworthy, that this method shows advantages over covalent oriented immobilization. Although, both methodologies have the advantage of obtaining an oriented antibody attachment for binding, in the case of metal-chelation immobilization the antibody is placed oriented perpendicular to the surface “end-on” orientation in contrast to the covalent immobilization where the antibody adopts a predominantly “flat-on” orientation, with the Fc and two Fab fragments lying flat on the surface.

### Example 7. Detection of Salmonella in a Real Food Sample

The advantageous antibody oriented immobilization shown in example 6, was tested for the detection of salmonella in a real sample. The result is reported in the FIG. 12, which illustrates the increment of temperature due to the salmonella in a real sample doped with a known number of Salmonella CFUs, in comparison with the signals generated by the negative controls.

The temperature increment, due to the presence of salmonella in the real sample on an oriented antibody immobilized microfluidic chip surface, was also higher than those obtained for the respective controls.

After building the calibration curve, the measurement of the increment of temperature due to the known different concentrations of salmonella and to the unknown concentration of pathogen in the real sample was determined, as reported in the FIG. 13.

The increment of temperature due to the presence of the theoretical number of CFU/ml used to dope the real sample, agrees with the number of CFUs of the calibration curve.

### Example 8. Sandwich Immunoassay for Campylobacter jejuni Detection: Capture Antibody Oriented Immobilization onto the Microfluidic Chamber Surface

The established protocol for the capture antibody oriented functionalization of microfluidic chamber, together with a sandwich immunoassay, was used for the detection of a pathogen different from Salmonella such as Campylobacter jejuni in order to demonstrate the universality of this technology.

Campylobacter jejuni is one of the four bacterial pathogens, together with Salmonella spp., Listeria monocytogenes (L. monocytogenes), and Escherichia coli (E. coli) O157:H7, estimated to account for approximately 67% of food-related deaths (Mead et al., 1999). Screening for Campylobacter is routinely carried out globally with different quantification methods which are available for the detection of this pathogen in food products, such as culturing, microscopy, enumeration methods and bio-chemical test PCR, immunoassays (Yang et al., 2013). Some of the aforesaid methods are sensitive and rapid but suffer from setbacks such are the fact that they are expensive, require extensive sample preparation, have poor selectivity and are time-consuming.

Indeed, as for salmonella, since most poultry-based products are consumed within days from the production date, this presents a challenge for available methods as while the method is being performed the population is exposed to Campylobacter leading to out breaks of food borne illness (Che et al., 2001).

The immunodetection of C. jejuni using HEATSENS in a microfluidic chip provides a cost-effective, rapid, easy, sensitive and reliable diagnostic approach.

C. jejuni was purchased heat-killed and lyophilized. They were re-suspended in PBS at different dilutions, and used to generate the calibration curve for further detection of an unknown sample (FIG. 14) in Bolton culture media.

The combination of HEATSENS technology and the antibody oriented functionalization of the microfluidic chamber surface, allows achieving low LOD (Limit of detection) of campylobacter in Bolton culture media.

Compared with the sensing of Campylobacter J reported by Masdor et Al. (Masdor et Al. Biosensors and bioelectronics 78, 2016, 328-336), which describes the development of a sensitive QCM sandwich immunoassay with a detection of 150 CFU/ml of Campylobacter, HEATSENS allows a detection of this specific bacteria pathogen lower than 100 CFU/ml.

Furthermore, this limit of detection is reached immobilizing 210 fold less capture antibody on the surface, decreasing the background and lowering the cost of production of the chip.

### Example 9. Sandwich Immunoassay for Ara h 1 Detection: Capture Antibody Oriented Immobilization onto the Microfluidic Chamber Surface

To further illustrate the universality of the present methodology we performed the present example with a still further analyte.

Peanuts (Arachis hypogaea) are one of the allergens most frequently associated with severe allergic reactions, including life-threatening food-induced anaphylaxis. According to the Food Allergen Labeling and Consumer Protection Act of 2004 (FALCPA 2004, Public Law 108-282, Title II) in the United States, and the Directive 2000/13/EC, as amended by Directives 2003/89/EC and 2007/68/EC, in the European Union, the presence of peanut in a food product has to be declared on its label.

The current reference method for detecting food allergens is the ELISA, even if there are also other analytical methods such as HPLC, capillary electrophoresis (CE), methods with laser-induced fluorescence (LIF) detection, enzyme linked immune affinity chromatography (ELIAC), size exclusion chromatography, and SPR. The determined LOD of ELISA is showed in the FIG. 15.

The combination of HEATSENS technology and the antibody oriented functionalization of the microfluidic chamber surface, allows to achieve lower LOD of Ara h1 in PBS using the same pair of capture and detection antibody (FIG. 16).

HEATSENS was thus successfully employed, in combination with oriented functionalized microfluidic surface in a bioassay to detect Ara h1.

The biosensor detection limit for Ara h1 was improved by one order of magnitude (LOD<0.4 ng/ml) compared with commercial ELISA kits (LOD Z10 ng/ml), and several orders of magnitude compared with other detection methods such as the SPR (J. Pollet et al./Talanta 83 (2011) 1436-1441).

### Example 10. HEATSENS in Microfluidic Applied to Other Analytes

The characterization of historic paints' binders still relies on conventional molecular biology methodologies that were developed decades ago and which have been substituted by more sensitive, specific, inexpensive and faster methodologies, taking advantage of the benefits of the emerging nanotechnology world.

HEATSENS was applied to the detection of collagen and albumin, two of the most used binders in pre-Renaissance paintings, illuminated manuscripts and sculptures in a microfluidic chip. This example again further illustrates the universality of the present methodology

1. Direct Immunoassay for the Detection of Albumin Absorbed onto a Microfluidic Chip Chamber Surface.

We implemented a direct immunoassay for the detection of Albumin. For this purpose, albumin as positive control (PC1), two micro-samples: one of albumin in powder from Zacchi® (sample 4) and another from glair painted on a glass surface exposed to the air for 1 year and a half (sample 5), samples were directly immobilized onto the microfluidic chamber surface. After the immobilization, the surface of the chip was blocked with milk in PBS 3 mg/mL, covering the chip surface, for 1 hour (at least) at 37° C. and shaking.

In order to test the specificity of the immunoassay, the following control experiments were performed: 1) NC1=absence of albumin and 2) NC2=absence on detection antibodies.

The results of the direct immunoassay, carried out in the microfluidic chip following the settle protocol, is depicted in FIG. 17.

The result demonstrates that HEATSENS, employed in combination with functionalized microfluidic surface in a bioassay, is able to detect Albumin in pigments. The present sensing methodology offers also the possibility of albumin quantification in complex matrices as the pigments are.

2. Sandwich Immunoassay for Detection of Collagen Using Capture Antibody Covalently Immobilized on Amicrofluidic Chip Surface

The detection of collagen, was also implemented by using an immunoassay in a sandwich format.

The capture antibodies were immobilized onto the microfluidic chip surface using the already described immobilization protocol, and two micro-samples: one from rabbit skin glue in water (10% w/w) (sample 4) and another micro-sample from a paint made by a mixture of glue+CaCO₃ painted over 40 years ago (real sample) that was recognized by the detection antibodies, were allow to flow inside the microfluidic chip.

In order to test the specificity of the immunoassay, the following control experiments were performed: 1) NC1=absence of collagen and 2) NC2=absence on detection antibodies.

The combination of HEATSENS technology and the antibody functionalization of the microfluidic chamber surface, allows to identify collagen in real samples.

The result of the collagen detection, using HEATSENS technology in a microfluidic chip is showed in the FIG. 18.

The result demonstrates that HEATSENS, employed in combination with functionalized microfluidic surface in a sandwich immunoassay, is able to detect collagen in pigments. The present sensing methodology offers also the possibility of collagen quantification in complex matrices as the pigments are.

### Example 11. Sandwich Immunoassay Protocol

The following protocol was found to be especially suitable for sandwich immunoassays using a microfluidic device and the Heatsens technology:
1. The channels are equilibrated by pumping washing buffer (BSA 0.5% in PBS1×, 0.1% tween) at a flow rate of 150 μlml for 5 minutes.
2. Then 1 ml of analyte sample is injected in the channel with a flow of 150 μl/ml.
3. After the sample, the channel is washed with washing buffer (BSA 0.5% in PBS1×, 0.1% tween), using a flow of 300 μl/min for 4 min.
4. The detection antibodies are injected in the channel. The flow is 150 μlml for 2.5 minutes.
5. After the detection antibodies, the channel is washed with washing buffer (BSA 0.5% in PBS1×, 0.1% tween), using a flow of 300 μl/mim for 4 min
6. The streptavidin@AuNPr are injected in the channel. The flow rate is 150 μlml for 2.5 minutes.
7. After the streptavidin@AuNPr, the channel is washed with washing buffer (BSA 0.5% in PBS1×, 0.1% tween), using a flow of 300 μl/min for 4 min and dried.

### Example 12. Extension of Oriented Functionalization Methodology to Other Material Surfaces

Oriented immobilization methodology through functionalization with metal-chelate on microfluidic chip can be extended to other types of surfaces such as metal (iron, cobalt, nickel, platinum, palladium, zinc, copper and gold), carbon (graphene, diamond, nanotubes, nanodots) and silicon surfaces. Grafting of diazonium aryl derivatives containing carboxylic groups can also be accomplished on these surfaces being a platform for a further stepwise functionalization with the metal-chelate layer.

It is also possible to functionalize other surfaces such as Polydimethylsiloxane (PDMS) and glass by covering the surface through self-assembly with organo-functional alkoxysilane molecules carrying a carboxylic acid function or epoxy groups. In this way, a study was carried out in a glass surface functionalized with epoxy groups by silanization and further introduction of a metal-chelate layer (NTA-Cu2+). The metal-chelate functionalized glass surfaces were assayed for both oriented and non-oriented covalent immobilization of biomolecule and employed the sandwich immunoassays to detect analytes in a sensitive way.

Simple glass surface modification was carried out in four steps. For the first step, the activation of glass supports was performed to remove all the organic residues in order to graft the epoxysilane on the surface. In the second step, the functionalization with epoxysilane was done with dry toluene to avoid gel formation of the silanes. The epoxy groups on the surface guarantee an efficient reaction with the amine group of the NTA at pH 10.8, where the amine of the NTA opens the epoxy group in a high molar ratio. And finally, in the last step, supports were incubated with 100 mM of CuSO4 in order to chelate the metal ion onto NTA moiety to orient the analyte.

Once glass slides were functionalized with NTA-Cu2+, an immunoassay for the detection of salmonella using HEATSENS technology was carried out (by using an oriented immobilization). For comparison, other methods of immobilization such as direct adsorption and covalent flat-on antibody immobilization were also assayed. The results of the different strategies of antibody surface functionalization are displayed in FIG. 19, where the increment of temperature due to the detected Salmonella in comparison with the generated background signal is shown for each surface functionalization.

This figure again demonstrates that the oriented immobilization of capture antibodies through metal-chelation provides the best results not only in terms of a high increment of temperature due to the presence of salmonella but also by providing a null signal due to nonspecific interactions (background). Thereby indicating the correct functionalization strategy as a crucial step to obtain an optimal antibody attachment in a favorable orientation, while avoiding nonspecific adsorption of HEATSENS labels (gold nanoprisms).

Glass surface functionalization is fast, easy, simple and inexpensive and can be used for different types of biomolecules.

### Example 13. Description of the Different Configurations of the Thermal Sensor Used for Measuring the Increment of Temperature Caused by the Presence of the Targeted Analyte

The important advantage of the sensing setup of HEATSENS using microfluidic chips for analyte capture is that all components are suitable for being assembled and miniaturized in a number of different ways, one of these being the one shown in FIG. 20.

Two possible configurations of the sensor system are mentioned below:

1. Thermal sensor behind sample; and

2. Thermal sensor in front of sample

In this sense, we can place the laser and thermopile (thermal sensor) in the same plane, with the thermopile pointing at the sample, tilted lightly upwards, or in different planes. The inclination is due to the saturation of the thermopile. When the laser beam irradiates directly to the thermopile, every temperature value reaches the maximum and the measurement is not valid. The thermopile has a FOV of 100×400 and the cameras, where the reaction takes place, are 3 mm high, 5 mm width. This results in an optimal distance between sample and thermopile of 17 mm to cover the camera; to ensure the measurement it is set at 20 mm.

If we place the laser and the thermopile in the same plane and we measure from behind, the sample is located with the thinner width next to the thermopile, so that the heat detected would not spread out and we can get the total information.

The results registered by using the configuration shown in FIG. 20 (the same plane), denote a linear increment of temperature related to an increment of the number of Salmonella CFUs, where the taken sample is consistent with the calibration curve (see FIG. 21).

However, the laser and thermopile can also be place in different planes. Once again the thermopile will be pointing to the sample, vertically tilted (≈40°) to avoid the laser irradiation (FIG. 22). The distance to the sample is also set to 20 mm where the thermopile can detect the heat increment of the specimen.

Measuring in front of the sample requires that the thinner width is on the thermopile and laser side. Here the laser irradiates in a focused manner, the light goes through a thinner part of the μfluidic chip and irradiates the sample.

In FIG. 23 the resultant curve is an exponential curve, the values concurred with the curve with an adj. R-Square equal to 0.99864. The saturation of the measurements is clearly visible. In this specific case the saturation was achieved in presence of very low concentrations of nanoprisms, due to the combination of the presented configuration and the behavior of the nanoprisms under laser illumination, as the limit of detection has increased compared to the previous disposal, achieving higher temperature increments for fewer CFU's.

The presented results were achieved using the Ventus laser system but considering the characteristics of the HEATSENS technology, also other NIR light source could be used such as the laser diode and LED.

### Example 14. Antibody Immobilization Methodology According to the Present Invention Versus Procedures Wherein Polystyrene Surfaces are Functionalized by UV Irradiation (185 nm), which Leads to the Generation of Carboxylic Groups

For sensing applications, the immobilization of the recognition biomolecule on the support where it occurs the sensing, must be as stable as possible, oriented and with a high-yield, to provide a high sensitivity to the sensing platform.

For HEATSENS sensing platform, as reported in the present specification, the chemistry has been modified for the specific oriented immobilization of capture antibodies used for the implementation of the sensing platform. There are two key factors for the oriented immobilization of antibodies on surfaces through the metal-chelation of NTA-M2+ to the histidine-rich metal binding site present in the antibody heavy chain (Fc):
1) The metal employed in the coordinative binding histidine residues.
2) The metal-chelate surface density for the antibodies successful oriented immobilization.

The first key factor of the HEATSENS sensing platform is the NTA-metal chelates employed. In this sense, we have assayed the immobilization of antibodies on gold nanoparticles functionalized with NTA-metal chelates: NTA-Cu2+ and NTA-Co2+ employing anti-HRP and anti-CD3, respectively, to demonstrate the unique methodology to be used to reach high sensitivity of the HEATSENS sensing platform.

The amount of immobilized antibody was calculated by measuring the protein remaining in the supernatant before and after every step in the immobilization process. Samples were withdrawn and analyzed by SDS-PAGE. Gels (12%) were used and stained with silver.

As it can be seen in the SDS-PAGE gels (FIG. 29), gold nanoparticles functionalized with NTA-Co2+ after incubation in antibody solutions, gave similar signals both inputs- and supernatant after immobilization (lanes 1,2) respectively for both gels, which indicates no attachment of antibody molecules for both antibodies. By contrast, in lanes 7 of both gels there is a signal of the antibody after immobilization, where in gel 1 there is not band appearing, and in gel 2 appears a band as a consequence of the full attachment of antibody molecules to gold nanoparticles functionalized with NTA-Cu2+.

The antibody immobilization on gold nanoparticles functionalized with NTA-Co2+ and NTA-Cu2+, was also evaluated by incubation with HRP and following measurement of its enzymatic activity. As it can be seen in FIG. 30, only the gold nanoparticles modified with NTA-Cu2+ chelate showed enzymatic activity after incubation with anti-HRP, confirming antibody immobilization. By contrast, negligible activity was observed for nanoparticles functionalized with NTA-Co2+.

The high affinity of antibodies for copper in comparison to other bivalent metals is also demonstrated using commercial strips of flat surface functionalized with copper ions and nickel ions (2D system). In this sense, antibody molecules against HRP were immobilized on these functionalized metal-chelate surfaces and the presence of captured HRP was quantified by a colorimetric immunoassay. As it is shown in FIG. 31A, NTA-Cu2+ functionalized surface showed a more intense yellow color associated to the activity of HRP. This indicates the presence of a higher amount of HRP enzyme captured and therefore antibody immobilized than NTA-Ni2+ functionalized surface. This is display even clearer in FIG. 31B by measuring the absorbance relative to the substrate of HRP at 450 nm. NTA-Cu2+ functionalized surfaces showed up to five times higher absorbance than NTA-Ni2+.

These results make evident the higher binding capacity of the antibodies oriented immobilized onto surface activated with copper chelate when compared to Ni. The same experiment has been carried out using asymmetric gold nanoparticles as label, for HEATSENS sensing detection (please refer to FIG. 32).

The higher antibody capture efficiency of the copper chelated surface is also established by using the HEATSENS detection methodology. In this sense, the increment of temperature nearly duplicated when 10 μg/mL of anti-HRP were immobilized on Cu ions in comparison to Ni ions.

All these results demonstrate the importance of the metal employed for the oriented immobilization of antibodies on surfaces through the metal-chelation of NTA-M2+.

Another key factor, that has a strong influence for the oriented immobilization of antibodies on surfaces through the metal-chelation of NTA-M2+ to the histidine-rich metal binding site present in the antibody heavy chain (Fc), is the metal-chelate surface density, which affects the yield of antibody immobilization. In order to demonstrate this fact, we performed an antibody-HRP immobilization study employing different coverages of NTA-Cu2+ using gold surface modified nanoparticles as a 3D system. These were obtained by varying the concentrations of EDC/sulfo-NHS as catalyzers for its incorporation.

Table 1 shows the enzymatic HRP activities of anti-HRP immobilized on gold nanoparticles functionalized with low and high surface coverages of NTA-Cu2+ and NTA-Co2+, respectively.

TABLE 1
Enzymatic activities of gold nanoparticles functionalized with
NTA-Cu2+ and NTA-Co2+ with low and high coverage
after incubation with anti-HRP and enzyme HRP.

| Enzymatic | | | |
|---|---|---|---|
| Activity | HRP input | AuNPs- | AuNPs- |
| (Abs/min) | Control | NTA-Cu²⁺ | NTA-Co²⁺ |
| Low | 0.027 | 0.0003 | 0.0003 |
| coverage | | | |
| High | 0.027 | 0.023 | 0.0024 |
| coverage | | | |

It is observed that only gold nanoparticles functionalized with a high surface coverage of NTA-Cu2+ after incubation with antibody-HRP gives practically full enzymatic activity associated to anti-HRP immobilization. On the contrary, no activity was observed for gold nanoparticles modified with a low concentration of NTA-Cu2+ or NTA-Co2+.

This demonstrates that antibody immobilization is significantly affected by the density of the metal-chelate on the surface, this being a crucial factor to be controlled.

The effect of the density of the active groups and of the metal on the immobilization of the capture antibodies onto the surface where the detection occurs, has also been evaluated on 2D surfaces. In this sense, we compared two different protocols. In particular, we used the protocol described in Chiu Wai Kwok et al, “In vitro cell culture systems for the investigation of the morphogen Sonic hedgehog (Shh), Dissertation, 16 Nov. 2011, wherein microchip surfaces were functionalized by UV irradiation (185 nm), leading to the generation of carboxylic groups.

We then formed the chelates by coordination of bivalent metals, such as Ni2+, with the carboxylic groups formed. The chelation was carried out using 40 mM NiSO2, which reacted with N2-N2-bis-(carboxymethyl)-L-lysine previously introduced via the amino terminal group on the COOH polymer surface. The metal modified surface was then used to immobilize a poly (6) hystidine tagged protein, in this specific case the ShhN protein.

Such protocol was compared to the HEATSENS surface functionalization, wherein in contrast to the above, the introduction of the carboxylic groups was accomplished by grafting of an organic layer using aryl diazonium salt chemistry and UV light (365 nm, 8 W); the carboxylic surface was then functionalized with 20 mM N2-N2-bis-(carboxymethyl)-L-lysine-25 mM CuSO4 via amidation catalyzed by 10 and 20 mM EDC sulpho-NHS, respectively. The two steps of chemical modification guarantee the creation of a homogeneous layer with a high density active groups.

Evaluation of the interfacial surfaces changes for both surfaces were characterized by Fourier Transform Infra-Red (FTIR) measurements. These were performed on a Spectrum One FT-IR Spectrometer equipped with the Universal ATR Sampling Accessory (Perkin Elmer).

The FTIR study of modified samples with NTA-Cu2+ functionalized by NIT procedure revealed the appearance of characteristic bands associated with the vibrational modes of amides (FIG. 33) and carboxylic groups of NTA in comparison with an untreated sample (FIG. 33). Absorption bands at 3420 and 3780 cm-1 correspond to N—H stretch of amide group and bands at 1609 and 1747 cm-1 were associated to stretch C═O group of carboxylic acid. By contrast, surfaces modified with NTA-Ni2+(FIG. 33) did not show absorption bands of amide group but NH-absorption associated to amine groups and negligible bands of carboxylic groups. Furthermore, the density of metal-chelates on the surface chip were quantified to know the NTA-Cu2+ for mm² for obtaining a sensing improvement. The quantification was carried-out by determining the concentration of the Cu2+ removed from the chelated surface using EDTA.

CuSO4 forms a chelate with NTA in order to orient the antibody, where the ratio COOH:Cu2+ is 3:1, so 1 mol of Cu2+ corresponds to 3 moles of COOH of NTA. UV-Vis spectra of five points of calibration curve are shown in FIG. 34, where absorbance is measured between 500 and 900 nm to see the corresponding peak of CuSO4. The spectrum of Cu2+-EDTA removed from the microfluidic chip surface is shown in FIG. 35A. A concentration of 4 μM Cu2+ is found by extrapolating the absorbance values on the calibration curve depicted in FIG. 34B. Considering that Cu2+ coordinates the three COOH groups of the NTA, 4 μM of CuSO4 coordinate 12 μM of COOH of NTA in a total surface area of 9.42 mm² of the microfluidic chip surface. Therefore, to have an optimal density of antibodies onto the flat surface the minimum concentration of chelating groups for mm² was found to be 1.3 M of COOH and 0.43 μM of Cu2+.

Similar experiments were carried-out with surfaces modified with NTA-Ni2+ by using the methodology reported in Chiu Wai Kwok et al giving negligible absorbance values and therefore undetectable Ni2+ amount. These results further confirm the lower yield of the chelate functionalization employing Chiu Wai Kwok et al technology rather than NIT technology.

Differences in the antibody immobilization and its binding capacity on modified surfaces depending on the protocols of surface functionalization (NIT and Chiu Wai Kwok et al) were also analyzed. Antibody against HRP was immobilized on NTA-Cu2+ and NTA-Ni2+ chelate functionalized surfaces. Their binding capacity of immobilized antibody to capture the HRP is demonstrated by measuring the activity of the HRP on the surface, determined by a colorimetric method as well as by Heatsens detection.

FIG. 36 shows the results of enzymatic activity of NTA-Cu2+(NIT methodology) and NTA-Ni2+ chelate (Chiu Wai Kwok et al methodology) functionalized surfaces after incubation with HRP. The result of the activity on both surfaces confirms the antibody immobilization, but the higher intensity of the absorbance determined on NTA-Cu2+ chelate modified surface than NTA-Ni2+ surface, demonstrates a higher yield of immobilization of antibodies as consequence of a high surface coverage of NTA-Cu2+. Therefore, the immobilization of the antibodies when carried out through the surface modification by the NIT methodology gives far better results than the one reported in Chiu Wai Kwok et al.

This result is further confirmed by carrying-out a HEATSENS assay. In this sense, FIG. 37 shows the increment of temperature due to the presence of biotinylated HRP, captured by the oriented immobilized antibodies on the two metals chelated surfaces. The measured increment of temperature results to be higher on the NTA-Cu2+ chelate modified surface compared with the increment of temperature measured on NTA-NI2+ surface, which again indicates a higher yield of antibody immobilization by using NIT methodology.

Other approach to demonstrate the differences of the protocols and strength of NIT methodology in comparison to Chiu Wai Kwok et al have been the detection of the pathogen Salmonella employing the high sensitivity of HEATSENS sensing platform. Immobilization of antibody was assayed on both surfaces functionalized with NTA-metal chelates: NTA-Cu2+(following protocol developed in NIT) and NTA-Ni2+(following protocol reported in document Chiu Wai Kwok et al); a total amount of 1000 CFU of salmonella interacted with the oriented immobilized capture antibodies. Once onto surface, the biotinylated-detection antibodies detected the pathogen enabling the interaction with streptavidin-HRP (for the colorimetric assay) or streptavidin-nanoprisms (for the HEATSENS assay). FIG. 38 displays the higher intensity of absorbance of HRP enzyme, relative to the presence of the analyte on surface functionalized with the protocol developed by NIT compared with the one functionalized with the protocol reported in Chiu Wai Kwok et al. As can be seen, the intensity of absorbance of the detection of the 1000 CFU of Salmonella on surface functionalized with NTA-Cu2+, is more than three times higher compared with the measured absorbance relative to the detection of the same amount of analyte onto NTA-Ni2+ functionalized surface.

The results of HEATSENS assay relative to the detection of Salmonella on differently activated surfaces, are displayed in FIG. 39. The results of the HEATSENS assay show the higher increment of temperature for the same amount of salmonella onto NTA-Cu2+ chelate surface than of the NTA-Ni2+. Moreover, the higher signal of the negative controls in the assay carried out onto NTA-Ni2+, compared with the positive assay, demonstrates the lack of effectiveness of surface functionalization using the protocol reported by Chiu Wai Kwok et al. The not homogeneous coverage of active groups onto surface could cause the non-specific interaction of the analyte and detection antibodies with surface. Besides, the signal of the negative controls in the assay carried out onto NTA-Cu2+ surface is three folds lower than the positive control, which makes the detection very effective. Moreover, their signal is lower than the negatives controls of the assay onto NTA-Ni2+ surface, indicating the better chemical functionalization of NTA-Cu2+ surface. The different protocol of surface functionalization offers higher coverage and homogeneity of the active groups onto surface, which results in an improved capacity of antibodies immobilization, and higher binding capacity, and a higher sensitivity of HEATSENS assay.

1. In vitro use of a kit or device comprising a microchip which in turn comprises a substrate, wherein said substrate comprises at least one channel in the substrate, the channel comprising an inlet, an outlet, and a flow-path connecting the inlet and outlet, wherein the inlet and outlet together define a midplane; and a portion of the flowpath travels transversely across the midplane, wherein the portion of the flowpath that travels transversely across the midplane includes a recognition site or sensing area having a modified surface comprising an oriented antibody, capable of detecting a target analyte, onto a chelating agent;
wherein the recognition site or sensing area having a modified surface with a chelating agent is made of a thermoplastic material functionalized with a diazonium aryl compound containing one or more carboxylic groups represented by formula II below: 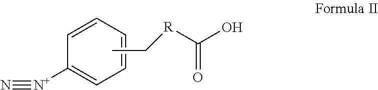
wherein R is an alkyl group having from 1 to 15 carbon atoms;
wherein the carboxylic groups resulting from the aforesaid functionalization with the formula II compounds are covalently linked to a chelating agent selected from the list consisting of Nα,Nα-Bis(carboxymethyl)-L-lysine hydrate (ANTA) metal (II) salt or nitrilotriacetic acid (NTA) metal (II) salt, wherein said metal (II) salt is understood as a salt of Cu2+;
wherein midplane is a plane passing through the channel in such a way as to divide it into symmetrical halves and wherein the sensing area is defined as the portion of the metal-chetale activated surface functionalized with the antibody, identified inside the flowpath that travels transversely across the midplane between the inlet and outlet;
for detecting an analyte as a result of the heat generated by metal nanoparticles when they are irradiated with an external light source.

2. The use according to claim 1, wherein the chelating agent is nitrilotriacetic acid (NTA) metal (II) salt and said metal (II) salt is understood as a salt of Cu2+.

3. The use according to claim 1, wherein the chelating agent is Nα,Nα-Bis(carboxymethyl)-L-lysine hydrate (ANTA) metal (II) salt and said metal (II) salt is understood as a salt of Cu2+.

4. The use according to any of claims 1 to 3, wherein the kit or device further comprises at least one of the following elements:
a. An external light source such as laser;
b. A second recognition molecule capable of recognizing the target analyte;
c. A metal nanoparticle with photonic properties; and
d. Optionally a device capable of detecting the heat generated by the metal nanoparticles when they are irradiated with the external light source.

5. The use according to any of claims 1 to 4, wherein the kit or device further comprises at least one of the following elements:
a. An external light source;
b. A metal nanoparticle with photonic properties functionalized with a second recognition molecule capable of recognizing the target analyte; and
c. Optionally a device capable of detecting the heat generated by the metal nanoparticles when they are irradiated with the external light source.

6. The use according to any of claims 1 to 5, wherein the kit or device further comprises at least one of the following elements:
a. An external light source;
b. A second recognition molecule (detection biomolecule) capable of recognizing the target analyte, optionally bound to a label molecule;
c. Metal nanoparticles with photonic properties functionalized with biomolecules specifically recognizing the detection biomolecule or the label with which the detection biomolecule is modified; and
d. Optionally a device capable of detecting the heat generated by the metal nanoparticles when they are irradiated with the external light source.

7. The use according to any of claims 4 to 6, wherein the kit or a device further comprises a device capable of detecting the heat generated by the metal nanoparticles when they are irradiated with the external light source selected from the list consisting of infrared cameras or thermopiles.

8. A kit or device comprising a substrate, wherein said substrate comprises at least one channel in the substrate, the channel comprising an inlet, an outlet, and a flow-path connecting the inlet and outlet, wherein the inlet and outlet together define a midplane; and a portion of the flowpath travels transversely across the midplane, wherein the portion of the flowpath that travels transversely across the midplane includes a recognition site for detecting a target analyte;
wherein the portion of the flowpath that travels transversely across the midplane that includes a recognition site is functionalized with the diazonium aryl compounds containing one or more carboxylic groups represented by formula II as defined in claim 1, and wherein the carboxylic groups resulting from the aforesaid functionalization are covalently linked to a chelating agent selected from the list consisting of Nα,Nα-Bis(carboxymethyl)-L-lysine hydrate (ANTA) metal (II) salt or nitrilotriacetic acid (NTA) metal (II) salt, wherein said metal (II) salt is understood as a salt of Cu2+.

9. The kit or device of claim 8, wherein the substrate is made of a thermoplastic material such as poly(methyl methacrylate), polystyrene, poly(dimethylsiloxane), polyethylene terephthalate, polyethylene, polypropylene, polylactic acid, poly(D,L-lactide-co-glycolide), or cyclic olefin copolymers, and comprises an antibody capable of recognizing the target analyte immobilized onto the recognition site or sensing area.

10. The kit according to any of claim 8 or 9, wherein the chelating agent is nitrilotriacetic acid (NTA) metal (II) salt and said metal (II) salt is understood as a salt of Cu2+.

11. The kit according to any of claim 8 or 9, wherein the chelating agent is Nα,Nα-Bis(carboxymethyl)-L-lysine hydrate (ANTA) metal (II) salt and said metal (II) salt is understood as a salt of Cu2+.

12. The kit or device of any of claims 8 to 11, which further comprises at least one of the following elements:
a. An external light source such as laser;
b. A second recognition molecule capable of recognizing the target analyte;
c. A metal nanoparticle with photonic properties; and
d. Optionally a device capable of detecting the heat generated by the metal nanoparticles when they are irradiated with the external light source.

13. The kit or device of any of claims 8 to 11, wherein the kit or device further comprises at least one of the following elements:
a. An external light source;
b. A metal nanoparticle with photonic properties functionalized with a second recognition molecule capable of recognizing the target analyte; and
c. Optionally a device capable of detecting the heat generated by the metal nanoparticles when they are irradiated with the external light source.

14. The kit or device according to any of claims 8 to 11, wherein the kit or device further comprises at least one of the following elements:
a. An external light source;
b. A second recognition molecule (detection biomolecule) capable of recognizing the target analyte, optionally bound to a label molecule;
c. Metal nanoparticles with photonic properties functionalized with biomolecules specifically recognizing the detection biomolecule or the label with which the detection biomolecule is modified; and
d. Optionally a device capable of detecting the heat generated by the metal nanoparticles when they are irradiated with the external light source.

15. The kit or device according to any of claims 8 to 14, wherein the kit or a device further comprises a device capable of detecting the heat generated by the metal nanoparticles when they are irradiated with the external light source selected from the list consisting of infrared cameras or thermopiles.
